(19) United States
(12) Patent Application Publication
(10) Pub. No.: US 2004/0033627 A1
(43) Pub. Date: Feb. 19, 2004

(54) Method and apparatus for detecting substances of interest

(75) Inventors: Turgut Aytur, Plattsburgh, NY (US); P. Robert Beatty, Berkeley, CA (US); Bernhard Boser, Berkeley, CA (US); Moshiur Anwar, Westminster, CA (US); Eva Harris, Berkeley, CA (US)

Correspondence Address: QUINE INTELLECTUAL PROPERTY LAW GROUP, P.C., P O BOX 458, ALAMEDA, CA, 94501, US

(73) Assignee: The Regents of the University of California

(21) Appl. No.: 10/453,846

(22) Filed: Jun. 2, 2003

Related U.S. Application Data
(60) Provisional application No. 60/384,630, filed on May 31, 2002.

(51) Int. Cl. G01N 33/553

(52) U.S. Cl. 436/526

(57) ABSTRACT

A method and/or system for detecting substances of interest. In specific embodiments, the invention involves a method and/or system using magnetic beads and easily manufactured electrical circuits to detect chemicals and/or substances of interest. In other embodiments, the invention involves a method and/or system for providing a variety of biologic assays. In further embodiments, the invention includes methods and/or systems for an associated device, referred to herein as a dual split-drain transistor.

### CROSS REFERENCE TO RELATED APPLICATIONS

This application claims priority from provisional patent application 60/384,630, filed May 31, 2002 and incorporated herein by reference.

### COPYRIGHT NOTICE

Pursuant to 37 C.F.R. 1.71(e), Applicants note that a portion of this disclosure contains material that is subject to copyright protection (such as, but not limited to, source code listings, screen shots, user interfaces, or user instructions, or any other aspects of this submission for which copyright protection is or may be available in any jurisdiction.). The copyright owner has no objection to the facsimile reproduction by anyone of the patent document or patent disclosure, as it appears in the Patent and Trademark Office patent file or records, but otherwise reserves all copyright rights whatsoever.

### FIELD OF THE INVENTION

The present invention relates to a method and/or system for detecting substances of interest.

### BACKGROUND OF THE INVENTION

The discussion of any work, publications, sales, or activity anywhere in this submission, including in any documents submitted with this application, shall not be taken as an admission that any such work constitutes prior art. The discussion of any activity, work, or publication herein is not an admission that such activity, work, or publication existed or was known in any particular jurisdiction.

Various strategies have been proposed for detecting substances and/or molecules and/or chemicals and/or compounds of interest. These strategies have been proposed for a number of applications such as, but not limited to, biologic assays and/or diagnostic tests, tests for drugs, explosives and/or other contraband substances, tests in manufacturing processes for desired or undesired substances, tests in food or manufacturing processes for contamination and/or pollution constituents, etc.

A number of strategies have been discussed that utilize magnetic and/or paramagnetic labels as part of a detecting device and/or system. In particular, various strategies have been discussed that utilize magnetic and/or paramagnetic beads coated with binding molecules in biological preparations and assays. Discussion of various of such strategies and related technology can be found in the below indicated patents and other publications.

U.S. Pat. Documents

| | | |
|---|---|---|
| 5240994 | Aug., 1993 | Brink et al. |
| 5318914 | Jun., 1994 | Matte et al. |
| 5372930 | Dec., 1994 | Colton et al. |
| 5445970 | Aug., 1995 | Rohr. |
| 5445971 | Aug., 1995 | Rohr. |
| 5558839 | Sep., 1996 | Matte et al. |
| 5580923 | Dec., 1996 | Yeung et al. |
| 5605662 | Feb., 1997 | Heller et al. |
| 5776748 | Jul., 1998 | Singhvi et al. |
| 6180418 | Jan 30, 2001 | Lee |
| 5981297 | Nov. 9, 1999 | Baselt |

Other References

| | | | |
|---|---|---|---|
| D.R. Baselt et | Biosensor Based on Force Microscope | J. V Sci. Technol. B, vol. 14, No. 2, | 1996 |
| al | Technology | pp. 789-793 | |
| R. Kotitz et al. | (41.sup.st annual conference on Magnetism and Magnetic Materials, see abstract | Nov. | |
| | book p. 73) | 1996 | |
| Lee et al., | Direct Measurement of the Forces | Science, vol. 266, pp. 771-773 | 1994 |
| | Between Complimentary Strands of DNA | | |
| P. Hinterdorfer | Detection and Localization of Individual | Proc. Natl. Acad. Sci. USA, vol. 93, | 1996 |
| et al., | Antibody-Antigen Recognition Events by | pp. 3477-3481 | |
| | Atomic Force Microscopy | | |
| G.U Lee et al., | Chemically Specific Probes for the | Israel J. Chem., 36, 8 1-87 | 1996 |
| | Atomic Force Microscope | | |
| M.N. Baibich | Giant magnetoresistance of (001) | Phys. Rev. Lett., vol. 61, No. 21, pp. | 1988 |
| et al., | Fe/(001) Cr magnetic superlattices | 2472-2475 | |
| U.M. | Magnetoresistive Memory Technology | Thin Solid Films, v. 216, pp. 162-168 | 1992 |
| Daughton, | | | |
| Fodor et al., | | Science v. 251, pp. 767-773 | 1991 |
| Kriz et al. | | Anal. Chem. 1996, 68, 1966-1970 | Jun. |
| | | | 1, |
| | | | 1996 |
| R. Kotitz, et al | SQUID based remnance measurements for | IEEE Trans. on Applied | 1997 |
| | immunoassays | Superconductivity, 7, 36 | |
| D. Baselt, et al | A biosensor based on magnetoresistance | Biosensors and Bioelectronics, 13, 791 | 1998 |
| | technology | | |
| R.L. Edelstein, | The BARC biosensor applied to the detection | Biosensors and Bioelectronics, 14, 805 | 2000 |
| et al | of biological warfare agents | | |
| R. S. Popovic | Hall Effect Devices | New York, IOP ltd | 1991 |
| Collins, M.L, | A branched DNA signal amplification assay | 1997 25 2979-2984 | 1997 |
| | for quantification of nucleic acid targets below | | |
| | 100 molecules/ml. | | |
| Herne, T.M. and | Characterization of DNA probes immobilized | J. Am. Chem. Soc. 1997. 119, p8916- | 1997 |
| M. J. Tarlov. | on gold surfaces. | 8920. | |
| Peterson, AW., | The effect of surface probe density on DNA | Nuc. Acids Research 2001 Vol.29 No.24 | 2001 |
| Heaton, R.J., | hybridization. | 5163-5168. | |
| Georgiadis, | | | |
| R.M. | | | |

Organization, P.A.H., Dengue and Dengue Hemorrhagic Fever in the Americas: Guidelines for prevention and control.
Vol. Scientific Publication #548. 1994.
Burke, D.S. and T.P. Monath, Flaviviruses, in Fields Virology, D.M. Knipe and P.M. Howley, Editors. 2001, Lippincott
Williams and Wilkins: Philadelphia. p. 1043-1126.
George, R. and L.C.S. Lum, Clinical spectrum of dengue infection, in Dengue and Dengue Hemorrhagic Fever, DJ. Gubler
and G. Kuno, Editors. 1997, CAB International: New York.
Vomdam, V. and G. Kuno, Laboratory diagnosis of dengue virus infections, in Dengue and Dengue Hemorrhagic Fever,
D. J. Gubler and G. Kuno, Editors. 1997, CAB International: New York.
Halstead, SB., Epidemiology of dengue and dengue hemorrhagic fever, in Dengue and Dengue Hemorrhagic Fever, D.J.
Gubler and C. Kuno, Editors. 1997, CAB International: New York.
Vaughn, D. W., et al., Evaluation of a rapid immnochromatographic test for diagnosis of dengue virus infection. J. Clin.
Microbio., 1998. 36: p. 234-238
Young, P.R., et al., An antigen capture enzyme-linked immunosorbent assay reveals high levels of the dengue virus protein
NS1 in the sera of infected patients. J Clin Microbiol. 2000. 38(3): p. 1053-1057.
Ghindlis, A., et al., Immunosensors: electrochemical sensing and other engineering approaces. Biosensors and
Bioelectronics, 1998. 13 (1): p. 113-131.
Kriz, C. B., K. Radevik, and D. Kriz, Magnetic permeability measurements in bioanalysis and biosensors. Anal Chem, 1996
68 (11): p. 1996-1970.
Baselt, D.R., et al., A biosensor based on magnetoresistance tehcnology. Biosens Bioelectron, 1998. 13 (7-8): p. 731-739.
Videa, E., et al., Recombinant polypeptides from the E protein of dengue virus types 1-4 for serological diagnoisi of dengue
infection. Manuscript in preparation.
Staropoli, I., et al., Affinity-purified dengue-2 virus envelope glycoprotein induces neutralizing antibodies and protective
immunity in mice. Vaccine, 1997. 15(17-18): p. 1946-1954.
Edeistein, R.L., et al., The BARC biosensor applied to the detection of biological warfare agents. Biosens. Bioclec., 2000.
14(10-11): p. 805-813.
Miller, M.M., et al., A DNA array sensor utlizing magnetic microbeads and magnetoelectronic detection. J Magnetism
Magnetic Materials, 2001. 225: p. 138-144.

Furthermore, diagnosis is an essential tool in the health care industry. The role of diagnosis is expanding, particularly within the context of screening and prevention. Infectious diseases are a major cause of death in the world, with HIV/AIDS, tuberculosis, and malaria responsible for approximately 5.7 million deaths in 1998. Rapid diagnosis is essential during epidemics for fast treatment and containment.

A dominant technology in diagnostics is the Enzyme-Linked Immunosorbent Assay (ELISA). Immunoassays, such as ELISA, are diagnostic tools that rely on the highly specific interaction of antibody binding to cognate antigen. In the ELISA, the detection of unknown antibody or antigen is signaled through an enzymatic label that activates a dye. ELISAs can be administered in a doctor's office or hospital environment, allowing for cost effective diagnosis of a broad range of diseases. However, most ELISA tests require a basic laboratory environment and some staff training. For many infectious diseases, a rapid, precise quantitative measurement is required for accurate diagnosis, which is rarely possible in the field.

### SUMMARY

The present invention relates to a method and/or system for detecting substances of interest. In specific embodiments, the invention involves a method and/or system using magnetic beads and easily manufactured electrical circuits to detect chemicals and/or substances of interest. In other embodiments, the invention involves a method and/or system for providing a variety of biologic assays. In further embodiments, the invention includes methods and/or systems for an associated device, referred to herein as a dual split-drain transistor. In further embodiments, the invention includes methods and/or systems for an alternative associated device, referred to herein as a micron scale Hall sensor. In further embodiments, the invention includes methods and/or systems for a field diagnostic detecting system.

The present invention, in specific embodiments, is involved with an improved sensor for magnetic bead detection. Previous magnetic bead detectors have involved one or more components that can be difficult and/or expensive to manufacture or use. Furthermore, such detectors generally do not effectively provide quantitation results using digital electronic circuitry. Thus, in specific embodiments, the present invention involves a magnetic bead detector that allows for quantitative electronic readout of detection of the presence of magnetic beads.

In further embodiments, the invention is involved with a detector for magnetic beads that includes an addressable array of detectors wherein addressing of a detector allows for a detection reading that can be used in a digital quantitation of an array detection result.

In further embodiments, it has been determined that a size (e.g., length and width) for an individually addressable detector element is preferably on the order of (e.g., within a factor of 10) the diameter of a magnetic bead used for detection and more preferable within a factor of 2-4.

In further embodiments, the present invention involves use of a small-scale Hall Effect detector (HD) to detect the presence of a magnetic bead. While the Hall Effect has been explored for over a century in detecting large scale magnetic fields, the present invention in specific embodiments involves a micron-scale Hall Detector. In further embodiments, a Hall Detector is integrated with addressable elements in an addressable array of Hall Detectors. In further embodiments, the addressable elements are used to rotate the drive and detection contacts of a four-contact Hall Device in order to provide an improved detection result. In further embodiments, a Hall Device is gated to allow the device to be deactivated and thereby allow compact device fabrication and/or shared row and column array addressing.

In further embodiments, the present invention is involved with a magnetic bead detector that can be manufactured using standard integrated circuit (IC) fabrication processes, such as well-known CMOS processes using silicon, other metals and/or semiconductors, or polymers. Creating a magnetic bead detector using standard electronic fabrication techniques allows for a detector and/or detector system that provides advantages in cost and manufacturability. Thus, in specific embodiments, the present invention, involves a CMOS sensor used as a magnetic bead detector that can be easily integrated with other electronic circuit functionality.

In further embodiments, the present invention involves paired or dual Hall Effect Devices to detect the presence of a magnetic bead. The novel configuration of dual devices provides for improved detection by allowing the devices to be compared to cancel out the effects of any large-scale or global magnetic field and thus improves detection of a local field generated by the presence of a magnetic bead or other small scale magnetic effects.

In further embodiments, the present invention involves a novel dual-channel, split drain transistor that can be used as a Hall Effect detector and in other applications.

In further embodiments, the present invention involves one or more magnetic bead detectors combined with other electronic circuit elements and/or mechanical elements to provide a detector system for magnetic detection. In specific embodiments, such a detector system is designed to be low-cost and disposable and to be used in conjunction with a reader system. In specific embodiments, such a detector system is embodied as a small-sized printed circuit board (PCB), providing an attachment for integrated components (e.g., a “flip-chip” configuration) and contacts for electrically connection to a reader and optionally also providing one or more container areas, such as wells, for holding a fluid or other substance on which detection will be performed. In specific embodiments, these container areas uses standard electrical component elements, such as solder, to provide fluid containment sealing.

In alternative specific embodiments, such a detector system is embodied as a large scale solid state integrated system where contacts and/or wells are fabricated using IC fabrication techniques, including etching techniques to create a containment area.

In alternative specific embodiments, a magnetic detector circuit is combined with wireless elements including an induction power source and a protective coating to provide a “smart dust” configuration wireless detector that can be added directly to a detection sample. In specific embodiments, the invention involves such a smart dust detector requiring a very low wireless reading range because samples containing the “smart dust” detectors are read in a portable reader such that wireless transmission elements of the reader are within one to a few millimeters of a sample containing the smart dust detectors.

In further embodiments, the invention involves systems and/or methods for detecting one or more diseases and/or disease conditions and/or other conditions of biological interest. Such a system will involve a reader as further described herein and one or more different specific binding molecules proximately fixed to a detector and may further involve one or more different binding molecules attached to magnetic beads.

In further embodiments, the invention involves systems and/or methods for performing biologic and/or medical assays in areas in particular that have little or no technological infrastructure. Such a system will involve a relatively low cost reader as further described herein and will further involve use of an off-the-shelf portable information appliance, such as a personal digital assistant (PDA). In specific embodiments, such a PDA is used to perform important clinical information gathering and recording from a reader, thereby allowing potentially sophisticated clinical data gathering even by relatively untrained personnel. In further embodiments, such an information appliance can further be used to perform one or more logic functions analyzing data from said reader to determine assay results, thereby enabling reduced overall system cost.

In further embodiments, the invention involves an immunoassay utilizing standard CMOS technology. In specific example embodiments, an array of Hall sensors is used to detect magnetic beads that serve as an assay signal. Electrical and magnetic modulation can be employed to improve the sensitivity of the sensors. In specific embodiments, devices according to the invention receive two post-processing steps to improve sensitivity and biocompatibility. In an example embodiment, a prototype devices according to the invention have been fabricated using a 0.25-μm BiCMOS process, and have successfully detected, for example, anti-Hu IgG antibody at a concentration of 200 pM.

While an example detector according to specific embodiments of the present invention is described herein as used for performing a biological assay, it will be understood to those of skill in the art that a detector according to specific embodiments of the present invention can be used in a variety of applications for detecting substances of interests. These applications include, but are not limited to: detecting pollutants in effluent from a manufacturing facility; detecting contaminants in foodstuffs; detecting the presence of a desired substance (such as petroleum components) in a mining or exploration operation; insuring the presence of desired elements in a manufacturing output.

Other Features & Benefits

The invention and various specific aspects and embodiments will be better understood with reference to the following drawings and detailed descriptions. For purposes of clarity, this discussion refers to devices, methods, and concepts in terms of specific examples. However, the invention and aspects thereof may have applications to a variety of types of devices and systems. It is therefore intended that the invention not be limited except as provided in the attached claims and equivalents.

Furthermore, it is well known in the art that systems and methods such as described herein can include a variety of different components and different functions in a modular fashion. Different embodiments of the invention can include different mixtures of elements and functions and may group various functions as parts of various elements. For purposes of clarity, the invention is described in terms of systems that include many different innovative components and innovative combinations of innovative components and known components. No inference should be taken to limit the invention to combinations containing all of the innovative components listed in any illustrative embodiment in this specification.

In some of the drawings and detailed descriptions below, the present invention is described in terms of the important independent embodiment of a biologic assay system. This should not be taken to limit the invention, which, using the teachings provided herein, can be applied to a number of other situations. All references, publications, patents, and patent applications cited herein are hereby incorporated by reference in their entirety for all purposes.

### BRIEF DESCRIPTION OF THE DRAWINGS

FIG. 1 illustrates an example process for a magnetic bead biologic assay applicable to specific embodiments of the present invention.

FIG. 2 illustrates a simulated magnetic field for a 5-μm magnetic bead in a 35-ka/m field, 5-μm for the surface, that can be used in understanding detection according to specific embodiments of the invention.

FIG. 3A illustrates basic operation of a split-drain Hall FET detector according to specific embodiments of the invention for a device that is scaled to be near the size of a detected magnetic bead.

FIG. 3B illustrates current lines for a device that is somewhat larger than a detected magnetic bead illustrating that according to specific embodiments of the invention the larger device has a reduced output signal due to charge redistribution.

FIG. 4A illustrates a sensor according to specific embodiments of the present invention comprising two hall devices with source current in opposite directions, reducing uniform fields by 30-40 db.

FIG. 4B illustrates an example of a sensor layout from a computer aided design (CAD) program according to specific embodiments of the present invention showing two channel regions, two sources at left and right sides of the sensor, and shared, split drains in the center of the sensor providing for source current in opposite directions.

FIG. 5 illustrates a block diagram of a gated Hall Device Sensor showing basic operation according to specific embodiments of the present invention.

FIG. 6 illustrates a block diagram of a gated Hall Device Sensor comparing gate and device operation to an FET according to specific embodiments of the present invention.

FIGS. 7A-D illustrates an example of a dual Hall Device Sensor layout from a computer aided design (CAD) program according to specific embodiments of the present invention and further illustrating rotational drive/detection according to further specific embodiments of the invention.

FIG. 8 illustrates an example of post-processing steps for fabricating a Hall Sensor for magnetic beads according to specific embodiments of the invention.

FIG. 9A illustrates a portion of the sensor array with 3-μm magnetic beads according to specific embodiments of the present invention wherein a Cr/Au layer was not applied to allow viewing of sensor detail.

FIG. 9B illustrates an example of a sensor array layout from a computer aided design (CAD) program according to specific embodiments of the present invention.

FIG. 10 illustrates relative data output of signal magnitude vs. array element index number according to specific embodiments of the invention.

FIG. 11 illustrates use of modulated (AC) magnetic and electrical drive signals and modulated output signal detection of a Hall sensor according to specific embodiments of the invention.

FIGS. 12A-B illustrate examples of simplified diagrams representing signal processing paths that can be performed in software and/or in hardware according to specific embodiments of the invention.

FIG. 13 illustrates an example simplified assembly diagram of sensor chip with fluid container and printed circuit board according to specific embodiments of the present invention.

FIG. 14 is a top-view image of an example sensor printed circuit board showing a sample well according to specific embodiments of the present invention.

FIG. 15 is a bottom-view image of an example sensor printed circuit board showing an attached ‘flip-chip” sensor array integrated circuit according to specific embodiments of the present invention.

FIG. 16 illustrates an example circuit schematic of a sensor according to specific embodiments of the present invention.

FIG. 17 illustrates a block diagram of an example portable reader assembly according to specific embodiments of the present invention.

FIG. 18 illustrates a block diagram of example functional components of an example portable reader assembly according to specific embodiments of the present invention.

FIG. 19 is a block diagram showing a representative example logic device in which various aspects of the present invention may be embodied.

FIG. 20 (Table 1) illustrates an example of diseases, conditions, or statuses for which at least one gene is differentially expressed that can evaluated according to specific embodiments of the present invention.

### DESCRIPTION OF SPECIFIC EMBODIMENTS

1. Overview

Micron-scale magnetic beads have been proposed and are in use in biologic applications, including for various clinical and research assays. Such labels have many advantages. For example, there are no comparable sources of magnetic signal in typical biologic system, so the background signal is intrinsically low. In addition, paramagnetic beads can be used to selectively manipulate biological systems by selective application of an external magnetic field. Such beads can be superparamagnetic, e.g., having very low remnance (the residual field after a magnetic field through it). When placed in a magnetic field, however, these beads generate an induced magnetic field. Many techniques are known for making magnetic beads biologically active. One technique used is to coat a polystyrene encapsulation of the beads with specific binding agents, such as specific binding molecules.

Assays utilizing magnetic labels including magnetic beads have been reported employed superconducting quantum interference devices as sensors. While these devices are highly sensitive to magnetic fields, they generally are not portable. Small scale sensors and sensor arrays have been proposed using a detection device based on giant magneto resistor (GMR) technology. GMR devices are highly sensitive, and are now in mass production in computer disk-drive read heads. In one proposed device, a metal surface (e.g., gold) over a GMR detector region receives a coating protein and a test liquid that might contain an antigen of interest is added under conditions that allow the antigen, if present, to bind to the coating protein. Next, magnetic beads coated with an appropriate antibody against the target antigen are added. Some type of washing is generally performed to remove beads that have not bound to the coating molecules. One proposed assay uses magnetic washing, where unbound beads are pulled from the sensor by a magnetic field. An external magnetic field orientated to the sensor surface is then applied. The induced field generated by bound beads is measured as a resistance change in a GMR sensor situated near the coating protein and the amount of change in resistance is measured. It is proposed that the amount of change corresponds to the number of beads bound and to use the resistance change in a sensor to quantitate the amount of target material. It has also been proposed to use a GMR sensor at a size near the size of a magnetic bead to detect a single bead and to use an array of such sensors. GMR sensors, however, have a number of disadvantages including the difficulty and costs associated with fabricating large numbers of them in an array using standard fabrication technologies.

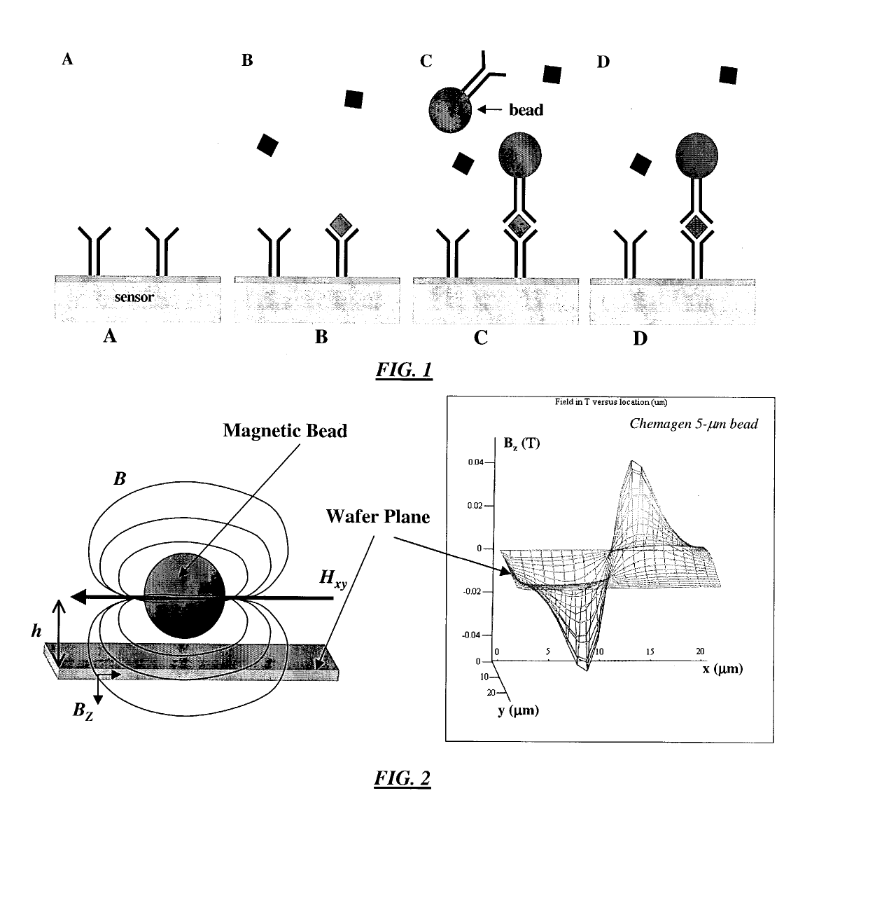

To provide a context for understand specific embodiments of the present invention, consider FIG. 1. FIG. 1 illustrates an example process for a magnetic bead biologic assay applicable to specific embodiments of the present invention. In this figure, a sensor is fabricated with a surface (such as gold) over it that can attach detector molecules (such as antibodies, proteins, oligonucleotides, or any binding molecule) of interest. It will be understood that while two detector molecules are shown, in an actual system a large number of detector molecules generally will be attached to the surface. A test liquid potentially containing a substance of interest is added (FIG. 1B) and as indicated in the figure some of the molecules may bind to the detector molecules. Subsequently or at the same time magnetic beads coated with an appropriate antibody against the target antigen are added (FIG. 1C). Though one representative detector molecule is shown, again a large number of molecules will generally be coated to the bead surface. After attachment, the device is washed (for example, either with a liquid or mechanically or magnetically) so that unbound beads are pulled away from the sensor (FIG. 1D).

Non-specifically-bound beads are the principle source of background in immunoassays. The effects of non-specific binding on assay performance have traditionally been reduced through liquid washing steps. Liquid washing has two disadvantages; it may not be practical in environments where adequate laboratory facilities are not available and it is imprecise in the removal of bound molecules. Specifically-bound molecules may be inadvertently removed, or non-specifically-bound molecules may be left, reducing assay sensitivity. In contrast, the magnetic beads used as the detection signal may by pulled from the surface by a magnetic field. This “magnetic washing” does not use viscous force to remove bound molecules, instead relying on magnetic force. Non-specifically-bound beads, where bond strength is low, are removed, while specifically-bound beads remain. Magnetic washing can be accomplished without removing sample fluid, and therefore does not require any deionized water. Furthermore, the magnetic field can be precisely controlled, and its control can be automated. Specific binding forces are typically greater than 50 pN, compared to less than 10 pN for non-specifically-bound particles, suggesting a gap that can be exploited to minimize non-specific binding. Magnetic washing was first described by Baselt et al and it was found that a 1 pN force removes 99.9% of non-specifically-bound particles.

Once the beads are attached to the device, they are generally placed in a global magnetic field that has a specific orientation to the surface of the detector. When using a paramagnetic bead, this field induces a local field at the bead, which is directed towards the sensor. An induced field generated by bound beads is then detected by the sensor.

2. A Practical Mallnetic Bead Sensor

While uses of biologically active magnetic beads have been recognized and small scale detectors have been discussed, previous proposals do not provide a practical means for constructing an inexpensive detector or system for performing assays. GMR devices remain expensive to fabricate using standard electronic integrated circuit fabrication techniques and practical systems and methods for detecting and/or quantifying substances of interest using such devices have yet to emerge.

According to specific embodiments, the present invention involves a magnetic bead sensor is constructed as an electronic circuit device that can be fabricated using standard microfabrication technologies, such as CMOS. In further specific embodiments, an active electronic device (e.g., a transistor or a Hall detector as herein described) provides greater sensitivity and enhanced functionality as herein described. It has also been found that ideally a sensor according to specific embodiments of the present invention will respond only to a field perpendicular to the device surface. Thus, according to specific embodiments of the present invention, selection of the device gives added immunity to effects from an excitation global magnetic field, which can be orientated parallel to a device surface.

According to further specific embodiments of the invention, the invention involves a sensor to detect the presence of a bound magnetic bead using the Hall Effect. The Hall Effect is a long recognized effect wherein a magnetic field perpendicular to an electric current will tend to deflect that current and this deflection can be measured as a voltage difference in a sheet conductor. The presence of this measurable voltage is called the Hall effect after E. H. Hall who discovered it in 1879. A typical Hall Device is a roughly square conductive surface with a current or voltage source connected between two opposite corners. A voltage difference measured between the remaining two corners has a proportional relationship to the strength of the magnetic field normal to the surface and is referred to as the Hall Voltage. Typical Hall Sensors are large-scale devices used to measure the strength and/or orientation of a magnetic field.

According to specific embodiments, the present invention involves a micro-fabrication scale device that uses the Hall Effect to detect the presence of a bound magnetic bead. Two such example devices according to specific embodiments of the invention are described herein referred to as “a Hall FET” and “a gated Hall sensor.” In specific embodiments, characteristics of such devices include that they can be fabricated entirely in CMOS or similar semiconductor fabrication technologies, that they can be easily integrated with other-electronic components, and that they can be activated and read using a standard row and column addressing.

3. Split Drain Hall FET

According to further specific embodiments, the present invention involves a sensor device that exploits the MOS transistor structure and is referred to herein as a dual-drain Hall FET. In general, Hall sensors can be understood as operating in current or voltage mode. General construction and operation of such a device is illustrated in FIG. 3A. As illustrated in the figure, this device can be understood as comprising a FET type device with a single source, a channel of width W and length L, and two drains as illustrated.

In a Hall device, a differential current between the sensing terminals can generally be expressed as:

i_{HALL}=μ_{H}GB_{z}I_{BIAS} (1)

where μ_{H} is the Hall mobility, G is a geometric constant determined from device dimensions that accounts for such things as current confinement at the device boundaries, I_{BIAS} is the driving current, and B_{z} is the normal magnetic field strength. A similar expression can be written for the Hall voltage.

For a HALL FET device according to specific embodiments of the invention, this equation can be written as:

$${i}_{d} = \frac{L}{2 W} {μ}_{H} G {B}_{z} {I}_{DS} \qquad ( 1 )$$

where the bias current is the drain/source current IDS and the differential current between the two drains i_{d} is the Hall current. In a particular embodiment, the two Hall FETs (NMOS or PMOS) are operating in saturation region, though in other embodiments they could be operating in linear region.

4. Gated Hall Sensor

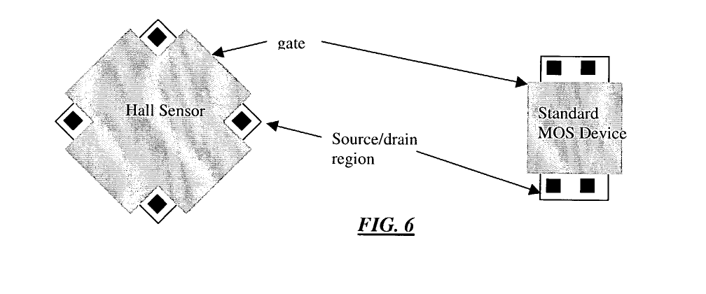

FIG. 5 illustrates a block diagram of a gated Hall Device Sensor showing basic operation according to specific embodiments of the present invention. FIG. 6 illustrates a block diagram of a gated Hall Device Sensor comparing gate and device operation to an FET according to specific embodiments of the present invention. A common configuration of Hall-Effect sensor consists of a resistive square with conductive contact made at each corner. As current is passed between two opposite corners, the presence of a magnetic field normal to the current flow causes deflection of the current. This current deflection then manifests itself as a voltage or current difference between the contacts normal to the current flow. According to specific embodiments of the invention, the resistive square is implemented as a Metal-Oxide-Semiconductor (MOS) device, where control of the voltage applied to a gate terminal determines the conductivity of the resistive square. By appropriately controlling this voltage, a particular sensor can be activated (sensitive to magnetic field) or deactivated (insensitive to magnetic field). This allows the common sensor configuration to be used in an array format where only selected elements are activated at one time.

The detailed construction of such a device according to specific embodiments of the invention is performed consistently with standard CMOS transistor fabrication. In a simplified description, it consists of four highly doped “source/drain” regions with metalized contacts for electrical connectivity. In the prototype device, these regions are n-type. These regions are implanted in a lightly doped tub or substrate, which is p-type in the prototype device. A thin oxide and polysilicon gate is defined between these contacts, forming a capacitor. When appropriate voltage is applied to the gate, a thin charge layer develops and allows conductivity and associated Hall-Effect sensing.

In further embodiments, both Hall FET and Hall Device sensors have a gate mechanism that can be used to activate the device. The optimum V_{gate} for particular devices is generally expected to vary based on specific device characteristics and can be determined empirically. For an example tested device, an optimum Vgate was determined to be approximate 1.5 to 2.5 volts. This generally provides the maximum signal and maximum signal to noise ratio. However, in some designs it may be desired to used lower voltages to reduce power consumption.

5. Sensor Scaled to Bead Size

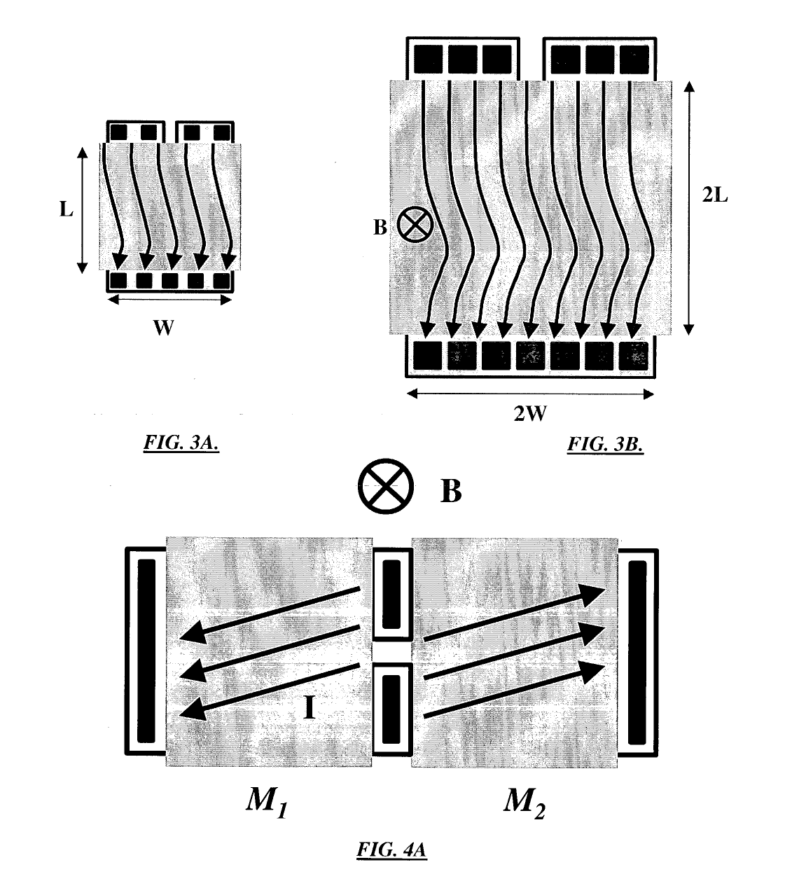

In further embodiments, the invention involves a Hall Effect sensor that is correctly scaled to detect the presence of a magnetic bead. In the analysis above, the applied magnetic field is assumed to be uniform and normal to the sensor surface. However, magnetic beads produce a local, non-uniform field when placed in an external field. It has been determined that this field is well modeled by a magnetic dipole equation. The peak signal decays with cubic dependence on the height from the sensor plane. A simulation of the induced magnetic field, measured normal to a plane 5 μm below the center of a 5-μm bead, is shown in FIG. 2.

The total field integrates to zero, since the field strength is anti-symmetric about the y-axis. Therefore, a hall-sensor device that is much larger than the bead will have a smaller signal than a correctly scaled device. Consider the hypothetical situation shown in FIGS. 3A and B. In these devices, the magnetic field is zero outside the shaded strip, and finite in it. The current bends in the region with field, as predicted, but tends to redistribute in the no field region. From this analysis, according to specific embodiments of the invention it has been determined that a Hall sensor device size should be scaled according the bead size.

Thus, according to specific embodiments of the present invention, matching the detector device size to the bead cross-section affects how well the device will work. There is a trade-off between selecting the size. Generally, it is desired to make the area of the sensing portion of the device close to and slightly less than the maximum cross-sectional area of beads used in the detecting assay, even if a particular fabrication technology being used would allow for smaller detectors to be constructed.

6. Example Dual Hall Sensors

According to specific embodiments of the present invention, the invention provides a Hall sensor constructed of dual Hall devices. According to specific embodiments of the invention, these devices are arranged so that any global magnetic field will generate Hall signals of opposite signs that add to zero in the dual device sensor. However, a local magnetic field the affects one device differently than the other, e.g., a field generated by an bound paramagnetic bead field scaled to about the size of one device, will produce a detectable non-zero signal in the subtracted Hall signals from the two devices. In one specific embodiments, a dual Hall FET sensor is used. In another embodiment, a dual Hall Device sensor is used.

Most HALL sensors are used for macroscopic magnetic field detection. According to specific embodiments, the present invention uses a unique configuration of dual Hall sensors, with current flowing in opposite direction to detect microscopic magnetic fields generated by magnetic beads. This configuration in not the same as using a reference device, because either or both devices could have a bead attached to it.

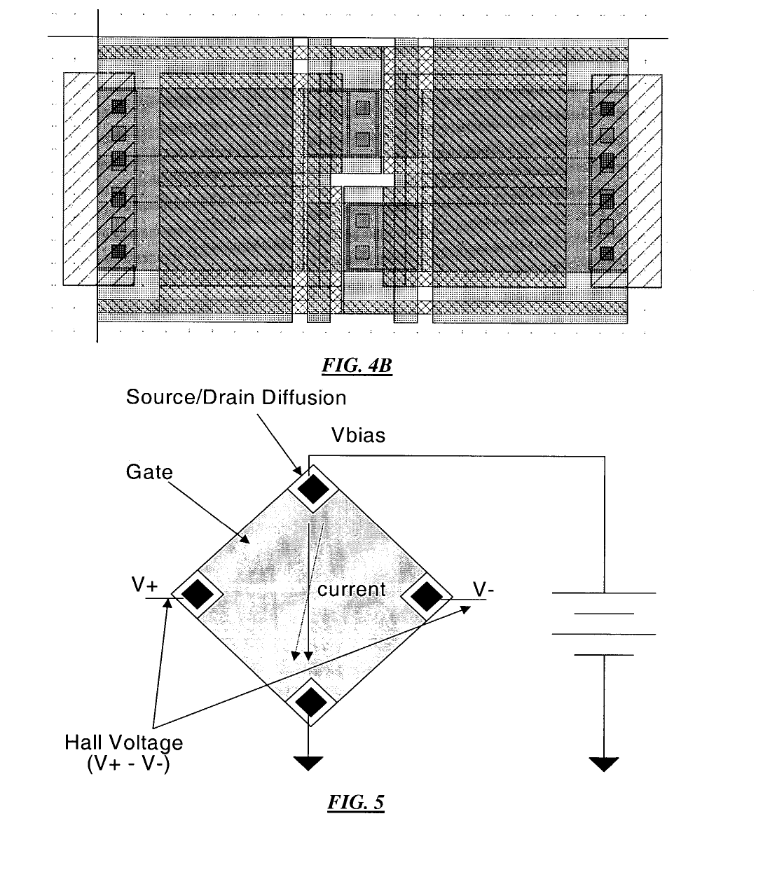

FIG. 4A illustrates an example design showing two Hall FETs that source current in opposite directions, thus reducing uniform field signals by 30-40 dB. In this design, each sensor consists of two matched devices that source current in opposite directions, so that uniform magnetic fields are rejected.

7. Example FET Sensor Fabrication

An example embodiment can be fabricated as a many sensor chip using known fabrication processes, such as an Agere Systems (formerly Lucent Technologies-Microelectronics) 0.25-μm single-poly 5-metal BiCMOS process. In this example, each Hall device is implemented as a 6-μm×6-μm dual-drain NMOS device. (PMOS devices can also be used.) The drains are separated by a 1-μm×1-μm field-oxide region. For a HALL FET embodiment, each sensor consists of two matched devices that source current in opposite directions so that uniform magnetic fields are rejected. FIG. 4B illustrates an example of a sensor layout from a computer aided design (CAD) program according to specific embodiments of the present invention showing two channel regions, two sources at left and right sides of the sensor, and shared, split drains in the center of the sensor providing for source current in opposite directions. In one example dual FET device, there are essentially five electrical contacts at each sensor, one for the gate, and one for each of the two sources and two drains. In alternative embodiments, the two sources are electrically connected and there are effectively four electrical contacts per sensor.

8. Example Hall Device Fabrication

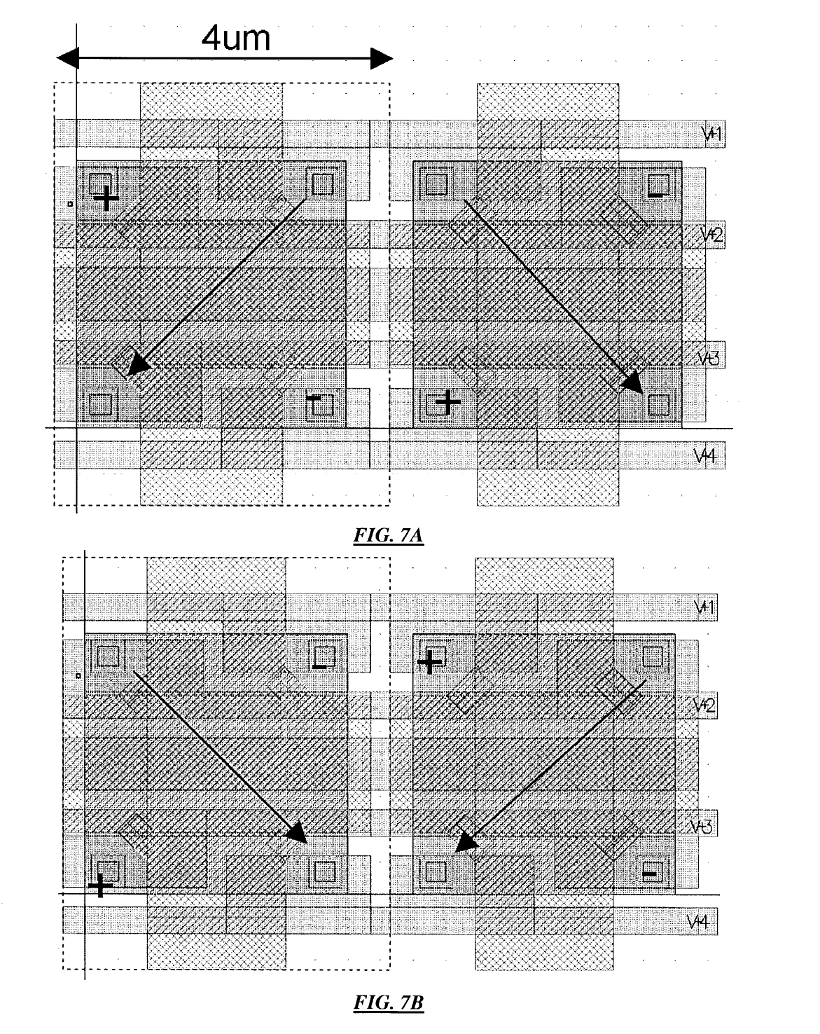

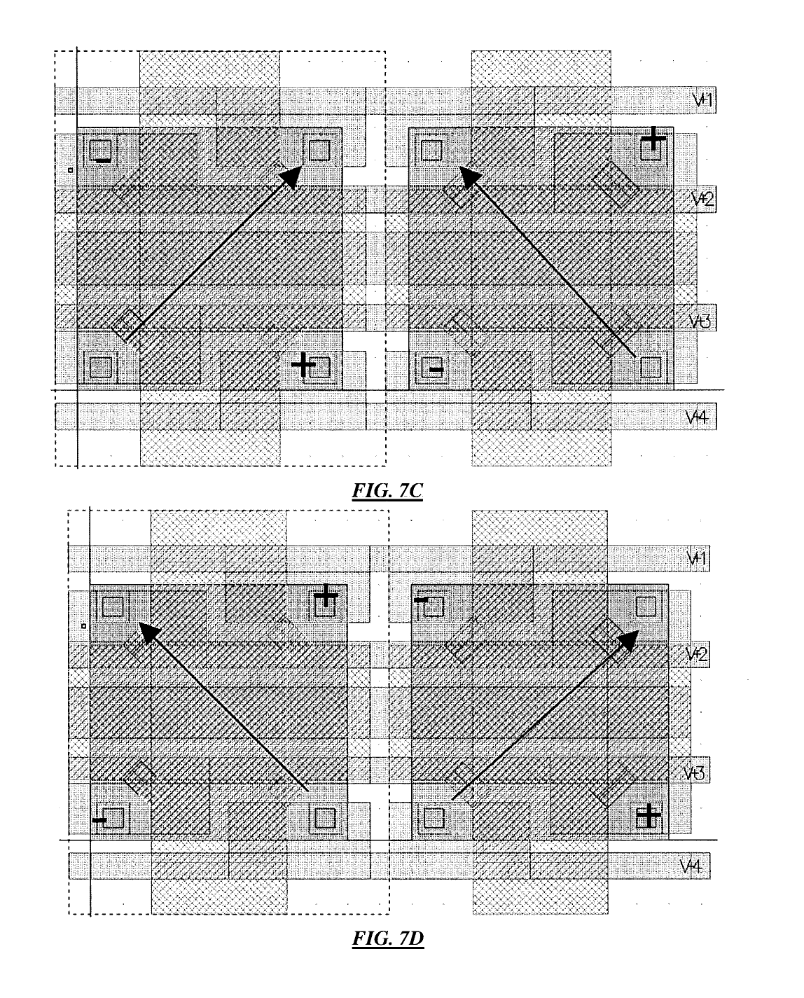

An further example embodiment can also be fabricated as a many sensor chip using one or more known fabrication technologies. For a Hall device embodiment, each sensor consists of two matched devices that subtract Hall signals in opposite directions so that uniform magnetic fields are rejected. FIG. 7A illustrates an example of a sensor layout from a computer aided design (CAD) program according to specific embodiments of the present invention showing two canonical Hall devices, each having four electrodes. The electrodes of the two devices according to specific embodiments of the invention are connected such that the inner upper two electrodes are each connected to a signal labeled as V1, the outer upper two electrodes are each connected to a signal a signal labeled V2, the outer lower two electrodes are each connected to a signal labeled V3, the inner lower two electrodes are each connected to a signal labeled V4. With this configuration, in the presence of a global magnetic field, a bias signal between V1 and V3 will produce roughly equal and opposite Hall signals between the V4 and V2 electrodes of each device. In the presence of a global field, these signals will sum to zero when measured between V4 and V2. However, a local magnetic field, such as produced by a bead of around 3-4 microns in diameter above one of the sensors will produce a detectable signal between V4 and V2. A gate voltage can be selectively applied to activate selected devices.

In further embodiments, the signal applications to V1-V4 can be rotated through four different cycles as shown in FIGS. 7A-D using solid state switching in an integrated circuit as known in the art and described herein and comparing and/or summing the net Hall signal from the dual device in each configuration gives improved sensitivity. While physically rotating a large scale Hall sensor to improve magnetic field strength detection is believed to have been previously discussed, using solid state switching in Hall devices is believed to be novel and using such with dual-Hall devices particularly novel.

In further specific embodiments, real-world dual device sensors may exhibit some net Hall signal even in the presence of no magnetic field. This signal can be measured before the exposure to any magnetic beads and thereafter compared and/or subtracted from the detector to determine a net signal.

In further embodiments, each sensor device on an array can be compensated using slightly different V_{gate} voltages for each half of the device. One way to do this is prior to use of the device to examine the differential voltage output of each sensor and then adjust the two Vgates until a desired differential Hall voltage (e.g., 0 volts) is reached, and then store the Vgate values for each sensor to be used later when selecting a cell. In various embodiments or applications, sensor cells may be looked at separately or may be examined by turning on an entire row and determining if any beads are present.

According to specific embodiments of the invention, one example Dual Hall device has been implemented using the National Semiconductor 0.25 um CMOS process. This process includes 5 aluminum metal layers and a single polysilicon layer. Metal 5 (top metal) is used as an etch mask for post processing, and metal 2 is used as an etch stop. Each hall sensor element measures 4 um×4 um, with 0.8 um source/drain diffusion areas at each of the four corners. Two such element are connected as noted previously to for each element of the array. Each column of an 32×32 element array is controlled by a polysilicon shared between each sensor in a column. Row decoding is implemented by a MOS switch array connected in series with the output nodes. These output of this switch matrix is connected to the post amplification circuitry. The direction of current is controlled by another switch matrix that allows for different connection configurations, as shown in FIGS. 7A-D.

9. Post Fabrication Detector Processing

Many commercially available CMOS fabrication processes today necessarily incorporate a large number of layers about the active devices. Five-layer CMOS processes, for example, may include five metal layers above the active devices to provide for interconnect.

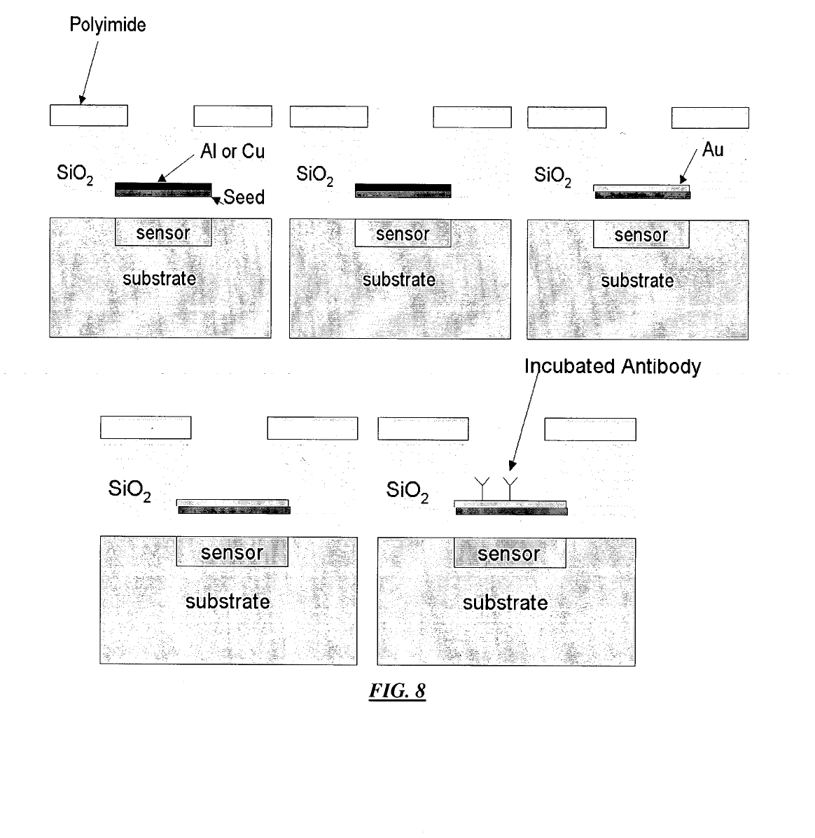

According to specific embodiments of the present invention, it is desirable to remove the majority of these layers in areas above the sensor array. It has been found that it is desirable to remove layers such that there is a very smooth surface that is as close to the sensing device as possible. FIG. 8 illustrates an example of post-processing steps for fabricating a Hall Sensor for magnetic beads according to specific embodiments of the invention. In an example method, this is done by first using a plasma or other etch down to an aluminum metal layer, and then etch the aluminum layer to expose a very smooth seed layer that was deposited on top of a chemically and/or mechanically polished oxide. In this example, a layer that can attach proteins (e.g., gold) is then deposited. It has been determined, according to specific embodiments of the present invention, that it is desirable to have a very smooth surface exposed to the magnetic beads in order to prevent beads being mechanically attached to bumps or ridges that would be present in a non-smooth surface.

In other fabrication processes, a copper layer near the detectors may be present from the outside fabrication process. In some cases, this layer may be smooth enough to be left after the initial etching and used for the protein binding layer.

10. Example Sensor Array

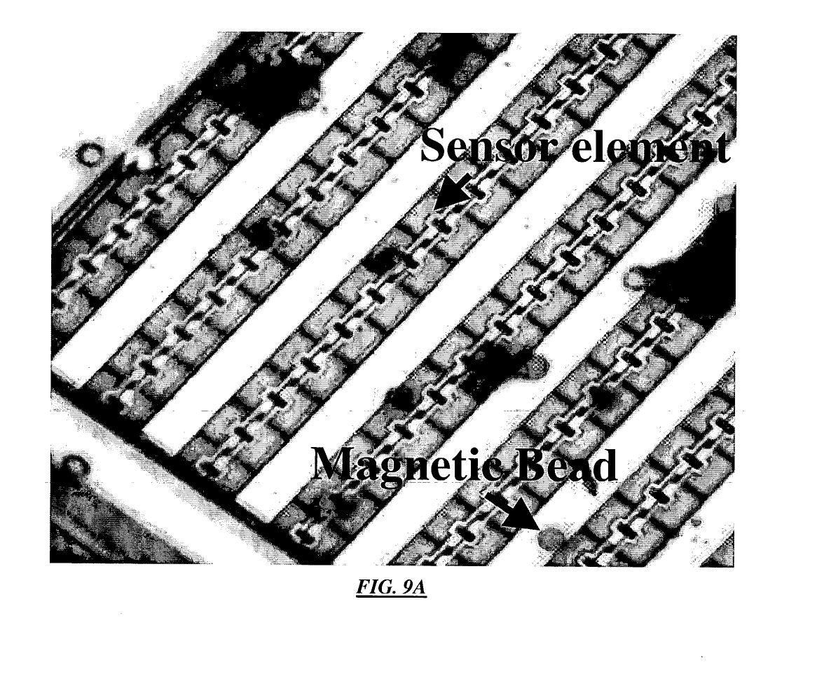

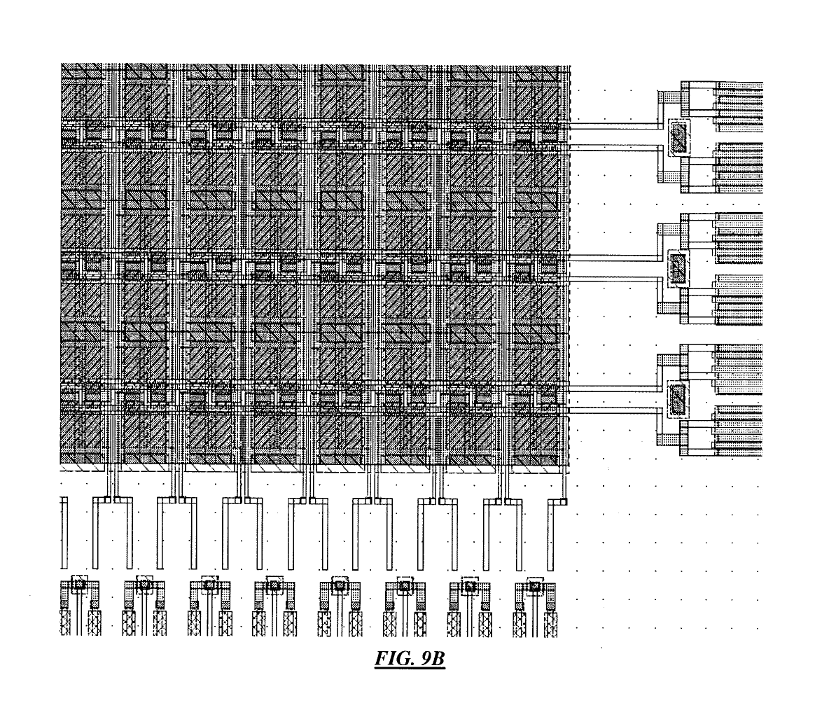

According to specific embodiments of the present invention, an example sensor array consists of a number of sensor elements, e.g., 32×8 or 32×32, etc.. FIG. 9A shows a portion of an example sensor array, with 3-μm magnetic beads bound over dual device sensors. The Cr/Au layer was not applied to allow viewing of sensor detail. In particular example arrays, each sensor element is addressable via a shift register. For example, a Hall current of a selected sensing device is converted to a voltage before being amplified by a bipolar tranconductance amplifier. This balanced, current-mode output is sent off chip. FIG. 9B illustrates an example of a sensor array layout from a computer aided design (CAD) program according to specific embodiments of the present invention.

Devices are post-processed in subsections of the initial 8″ wafer. First, the silicon dioxide above the sensor area is thinned by plasma etching. This reduces the distance from a magnetic bead to the sensor surface, increasing the signal. The etch depth is controlled by comparison with etch reference marks implemented in the standard metallization, resulting in a final oxide thickness of approximately 2 μm. Next, a thin layer (50 nm/150 nm) of Cr/Au is patterned over the sensor area using lift-off. Other materials were tested for protein adsorption, including Ti and Cu, but were found to be less effective than Au. However, Cu may represent a significant advantage in processing simplicity for CMOS processes that use Cu for metalization.

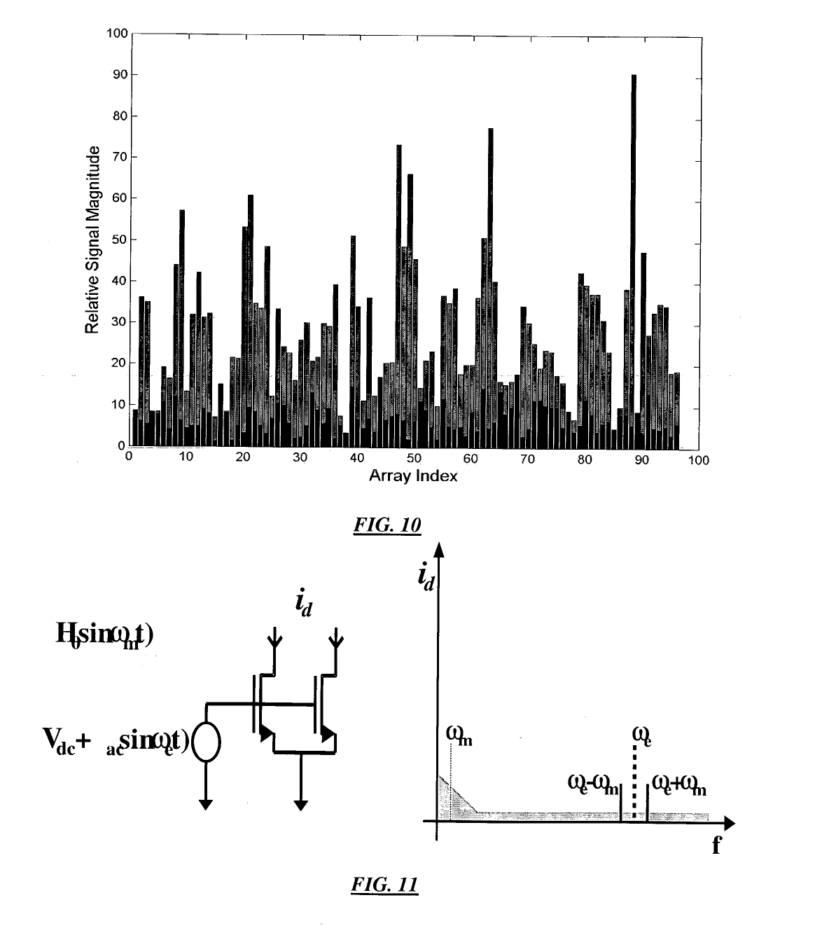

FIG. 10 illustrates relative data output of signal magnitude vs. array element index number according to specific embodiments of the invention As seen in the figure, depending on a threshold determination, a positive/negative signal result can be determined for each sensor in the array and the total number of results can be counted to provide a quantitation.

11. AC Excitation And Detecting Signal Processing

Generally, CMOS devices have poor low-frequency noise properties due to flicker or 1/f noise. The noise spectral density, as the name suggests, is inversely proportional to frequency. The consequence of this is that signals at or around DC do not can be difficult to detect as the effective noise increases as frequency decreases to zero. Furthermore, these signals do not benefit from narrowing of the noise bandwidth, eliminating the trade-off between bandwidth and Signal-to-Noise Ratio (SNR). Thus, there exists at D.C. a minimum detectable signal, independent of noise bandwidth.

For conventional magnetic sensing applications, e.g., using Hall sensors, the magnetic signal frequency is either not known or assumed to be at DC. However, according to specific embodiments of the invention, the excitation frequency of the external magnetic field is limited only by practical constraints of electromagnets. The invention in specific embodiments uses this by exciting paramagnetic beads at some frequency (e.g., around 2 KHz) and optionally also driving the bias signal at a different frequency (e.g., around 17 KHz). This can be used according to specific embodiments of the invention to improve the signal detection in various ways: For example, a band-pass filter can be employed to restore the trade-off between bandwidth and SNR. Also, the signal can be moved to a frequency of lower spectral noise density.

In general, however, there is still approximately one order of magnitude difference between the flicker noise corner frequency of the MOS hall device and the maximum frequency to practically operate the electromagnetic excitation. To overcome this, according to specific embodiments, the present invention combines electrical modulation and magnetic modulation, as represented in FIG. 11.

In particular embodiments, the gate-to-source voltage and magnetic field can be applied as:

V_{gs}(t)=V_{DC}+V_{AC} sin(ωₑt)

H_{xy}(t)=H₀ sin(ωₘt)

Where ωₑ and ωₘ are the electrical and magnetic modulation frequencies, respectively. Combining these equations with the Hall equations described above defines the modulated output spectrum. In addition to DC and higher order terms, the Hall signal (e.g., a differential drain current, Hall current, or Hall voltage) will contain the term:

Hall_signal∝ sin(ωₑ±ωₘ)

The electrical modulation frequency can be selected such that thermal noise, rather than flicker noise, dominates. The magnetic modulation is desirable to separate the wanted signal from carrier leakage (e.g., the dashed line in FIG. 11). Carrier leakage results from a variety sources, including device leakage and parasitic coupling, and results in a limit on the minimum detectable signal.

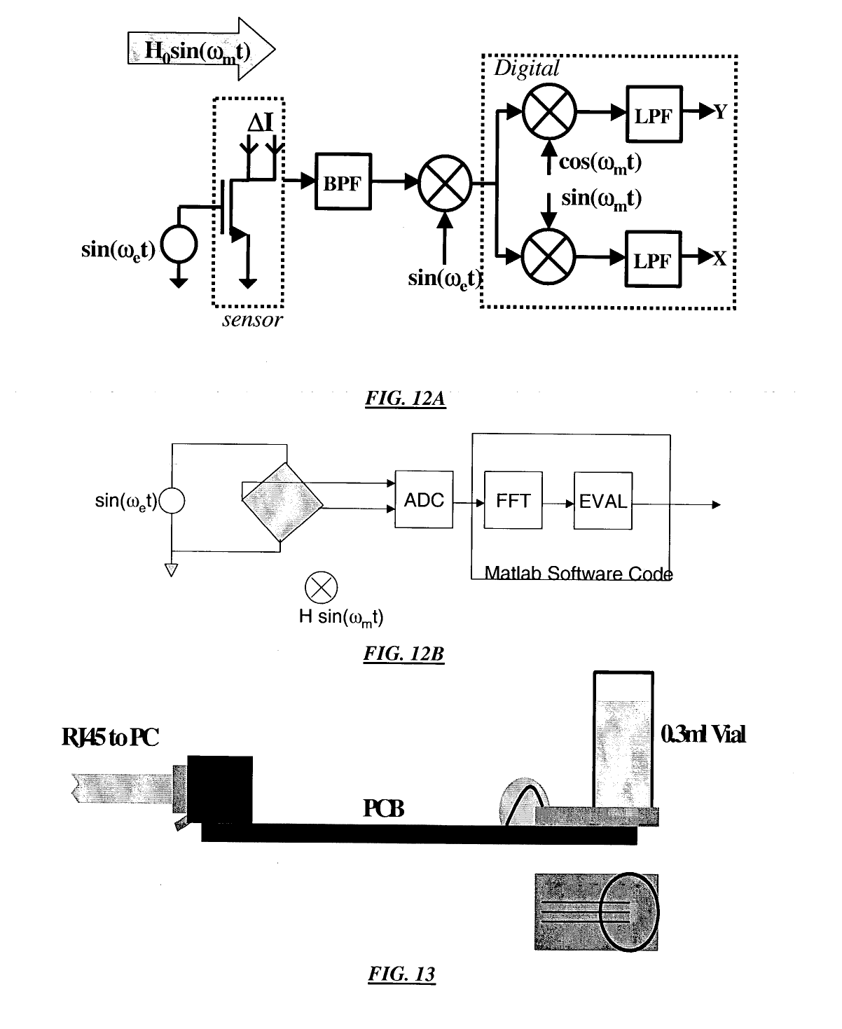

Simplified diagrams of the signal processing are shown in FIGS. 12A-B. In a specific embodiment, data is processed automatically in Matlab (The Mathworks, Natick, Mass.). The signal is digitally demodulated into in-phase and quadrature baseband components. These baseband signals are filtered by a FIR low-pass filter before reconstruction into polar form. The noise bandwidth can be adjusted by controlling the FIR filter bandwidth.

12. Example Source Code

According to specific embodiments of the invention, one or more signal processing functions and one or more array addressing and/or data capture and/or other functions are performed using logical instructions that execute on a stored-program logic execution device such as a personal computer, ASIC, PDA, etc. In a specific applications, these functions are specified in a Matlab programming language, as will be understood in the art. Various exemplary Matlab code modules are provided below. These code modules are provided as examples only and only some or none of these specific modules will be used in specific implementations.

RUN1.M
function dataS=run1(filename)
y=wavread(filename);
diff_y= y(:,1)−y(:,2);
dataY=parse2(diff_y);
dataS=getSideband(dataY);
RUN2.M
function dataS=run2(filename)
y=wavread(filename);
diff_y= y(:,1)−y(:,2);
dataY=parse3(diff_y);
dataS=getSideband(dataY);
PARSE.M
function dataP=parse(data)
SAMPLE_WIDTH=11025;
HEADER=1025;
START=30500;
data=data(START:length(data));
%parse
NUM_ELEM=floor(length(data)/SAMPLE_WIDTH);
dataP=zeros(SAMPLE_WIDTH-HEADER,NUM_ELEM);
for i=0:NUM_ELEM-1;

dataP(:,i+1)=data(i*SAMPLE_WIDTH+HEADER+1:

i+1)*SAMPLE_WIDTH);

end
PARSE2.M
function dataP=parse(data)
SAMPLE_WIDTH=11025;
WINDOW_WIDTH=500;
HEADER=1025;
START=0;
THRESHOLD1=2e−3;
THRESHOLD2=8e−4;
%first find max, then check power
index=1;
delta_data=data(2:length(data))−data(1:length(data)−1);
while START= =0,

```
[y,i]=max(abs(delta_data(index:index+WINDOW_WIDTH)));
if y>THRESHOLD1,
  sum(abs(delta_data(index:index+WINDOW_WIDTH)))
  /WINDOW_WIDTH
```

if sum(abs(delta_data(index:index+WINDOW_WIDTH)))

/WINDOW_WIDTH>THRESHOLD2,

START=i+index

end

end
index=index+WINDOW_WIDTH;

end
data=data(START:length(data));
%parse
NUM_ELEM=floor(length(data)/SAMPLE_WIDTH);
dataP=zeros(SAMPLE_WIDTH-HEADER,NUM_ELEM);
for i=0:NUM_ELEM-1;

dataP(:,i+1)=data(i*SAMPLE_WIDTH+HEADER+1:(i+1)
*SAMPLE_WIDTH);

end
PARSE3.M
function dataP=parse(data)
SAMPLE_WIDTH=11025;
WINDOW_WIDTH=500;
HEADER=1025;
START=0;
THRESHOLD1=2e−3;
THRESHOLD2=5e−4;
FMOD=0.4;
f1=round(FMOD*WINDOW_WIDTH)+1;
%first find max, then check power
index=1;
delta_data=data(2:length(data))−data(1:length(data)−1);
while START= =0,

temp_fft=abs(fft(blackman(WINDOW_WIDTH)
.*data(index+1:index+WINDOW_WIDTH)));
if temp_fft(f1)> 2*mean(temp_fft),

START=index+WINDOW_WIDTH

end
index=index+WINDOW_WIDTH;

end
data=data(START:length(data));
%parse
NUM_ELEM=floor(length(data)/SAMPLE_WIDTH);
dataP=zeros(SAMPLE_WIDTH-HEADER,NUM_ELEM);
for i=0:NUM_ELEM-1;

dataP(:,i+1)=data(i*SAMPLE_WIDTH+HEADER+1:(i+1)
*SAMPLE_WIDTH);

end
GETSIDEBAND.M
function P = getSideband(dataP);
FMOD=0.4;
FMAG=0.045;
DATA_LENGTH=length(dataP(:,1));
f1=round((FMOD−FMAG)*DATA_LENGTH+1);
f2=round((FMOD+FMAG)*DATA_LENGTH+1);
for i=1:length(dataP(1,:)),

tempFFT=abs(fft(hamming(DATA_LENGTH).*datap(:,i)));
P(i)=(tempFFT(f1)+tempFFT(f2))/2;

end
GETMEAN.M
function [meanP, varP]=getMean(dataP);
FMOD=0.4;
FMAG=0.045;
f1=round((FMOD−FMAG)*length(dataP(:,1))+1);
f2=round((FMOD+FMAG)*length(dataP(:,1))+1);
for i=1:length(dataP(1,:)),

tempFFT=abs(fft(dataP(:,i)));
P(i)=(tempFFT(f1)+tempFFT(f2))/2;

end
meanP=mean(P);
varP=var(P);
DEMOD1.M
function P = demod1(dataP);
FMOD=0.4;
FMAG=0.04;
t=1:length(dataP(:,1));
sin1=sin(2*pi*(FMOD−FMAG)*t)′;
cos1=cos(2*pi*(FMOD−FMAG)*t)′;
sin2=sin(2*pi*(FMOD+FMAG)*t)′;
cos2=cos(2*pi*(FMOD+FMAG)*t)′;
for i=1:length(dataP(1,:)),

X1=mean(sin1.*dataP(:,i));
Y1=mean(cos1.*dataP(:,i));
X2=mean(sin2.*dataP(:,i));
Y2=mean(cos2.*dataP(:,i));
P(i)= (X1\hat{ }2+Y1\hat{ }2)\hat{ }.5 + (X2\hat{ }2 + Y2\hat{ }2)\hat{ }.5;

end

13. Example Packaging and Example Applications

According to specific embodiments of the present invention processed chips, including metallization extension can be assembled as drawn in FIG. 13, which illustrates a simplified assembly diagram of sensor chip with fluid container and printed circuit board which can be used particularly in an experimental setup. In a specific embodiment, 9 mm×15 mm chips are mounted on a 15-cm long, PCB. A 300-μL polystyrene vial is inverted and epoxied to the silicon chip. A small hole is predrilled in the vial to allow fluid entry. The PCB connects to processing circuitry via a connector, such as an RJ-45 edge connector.

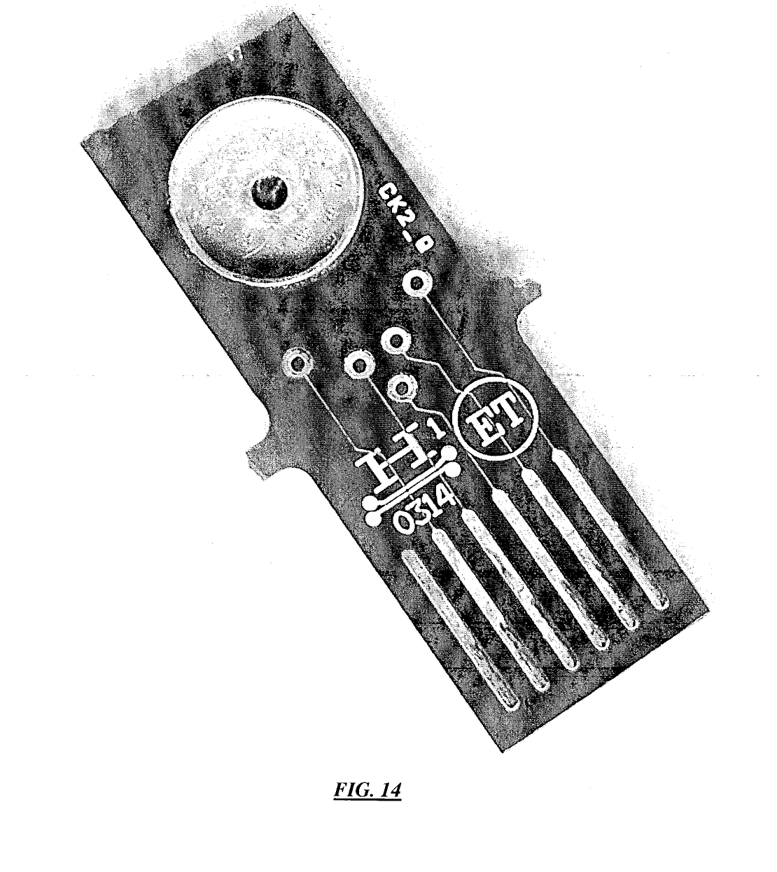

FIG. 14 is a top-view image of an example sensor printed circuit board showing a sample well according to specific embodiments of the present invention. The dimensions of this example board are roughly 2 cm long×0.35 inches wide×0.75 mm thick. The circular well is about 50 mm deep with a capacity of 70 micro liters. All of these dimensions are given as examples only, and other implementations are possible.

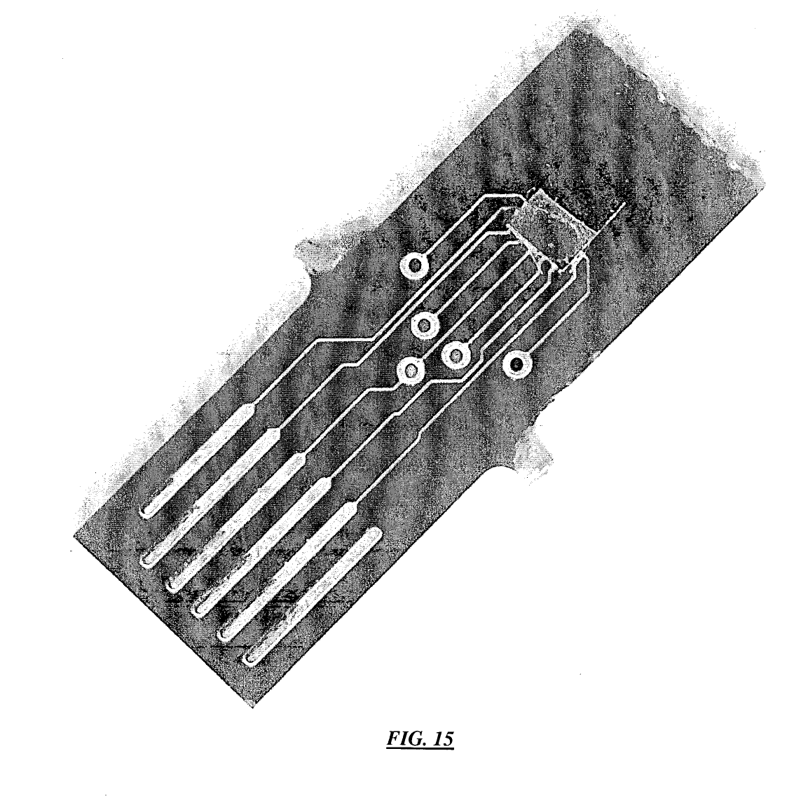

FIG. 15 is a bottom-view image of an example sensor printed circuit board showing an attached ‘flip-chip.” sensor array integrated circuit according to specific embodiments of the present invention. It will be seen that in this example both surfaces have six electrical contacts at one edge that allow for electrical connection with a reader.

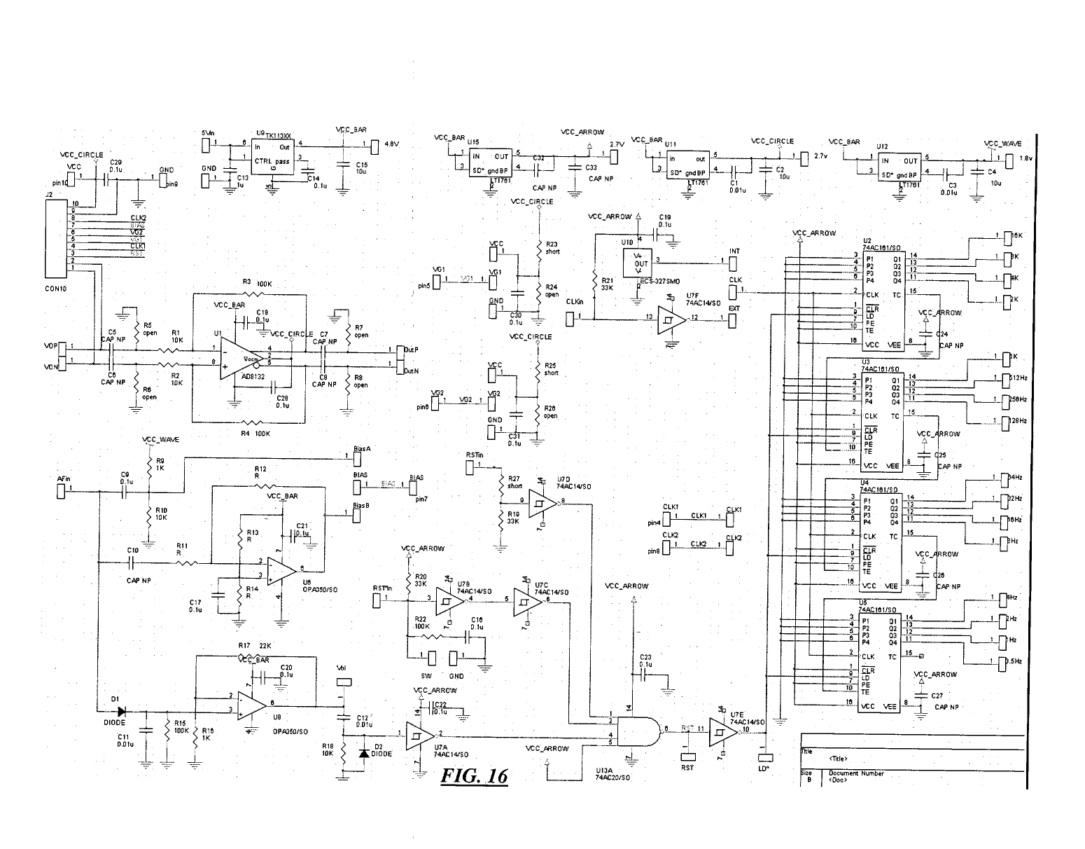

FIG. 16 illustrates an example circuit schematic of a sensor according to specific embodiments of the present invention.

14. Example Portable Reader

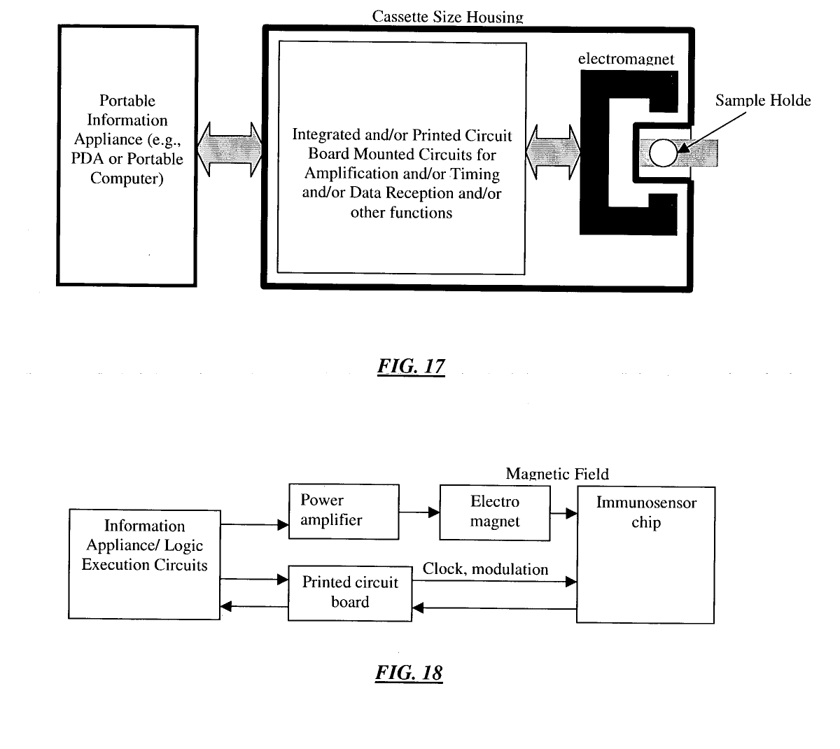

FIG. 17 illustrates a block diagram of an example portable reader assembly according to specific embodiments of the present invention. An example reader is further designed to be used with an information appliance, such as a laptop or personal computer or personal digital assistant (PDA) optionally with audio-band inputs and outputs.

An example reader is designed to be approximately audio cassette sized, or palm-sized and comprise electronic circuitry including amplification and timing circuitry, an electromagnet, and an opening for receiving a sample holder. In an example system, either the reader or the connected information appliance produces two sinusoidal outputs using audio ports, such as a 2-kHz signal for electromagnet and a 15 kHz-250 kHz for electrical modulation. The 2-kHz signal is sent to an audio power amplifier type circuit and then to the electromagnet. The electrical modulation output is either connected directly to the sensor chip or to a buffer amplifier first. In some prototypes, an additional signal of 10 kHz-100 kHz is applied to the sensor chip to rotate the direction of current flow. The sensor outputs are connected to reader circuitry for amplification, and then optionally to the audio-port inputs of the information device. A 1-10 Hz clock can be used to control the incremental sampling of each sensor element in the sensor array. The incoming signal is digitized by an analog-to-digital converter either in the reader on the portable information appliance. The digitized signal is processed using logic routines, such as the example matlab code supplied herein. In one example processing, first, the signal stream is parsed into data output from each sensor element. Next, a windowed FFT is applied. Finally, the energy of the appropriate spectral bins is added and compared against a threshold. Is some prototypes, the sensor chips are first calibrated by measuring each sensor element in the sensor array for signal response prior to use in the assay. This allows a baseline reference for signal comparison. FIG. 18 illustrates a block diagram of example functional components of an example portable reader assembly according to specific embodiments of the present invention.

15. Example Experimental Results

Anti-Hu IjG Assay

In a particular experiment, the sensor chip surfaces were coated overnight with 10 μg/ml human IgG diluted in phosphate buffered saline (PBS). Surfaces were blocked with 3% Non-Fat Dry Milk for 1 hour and washed 3 times in PBS with 0.5% Tween-20 (PBS-T). Either biotinylated goat anti-human IgG (200 pM) or biotinylated goat anti-mouse IgG (as a control) (Sigma Aldrich, St. Louis, Mo.) was added to separate vials and incubated for 30 minutes. The samples were then washed 3 times with PBS and streptavidin-coated magnetic beads (5-8 um diameter, Chemegen, Germany) diluted 1:200 to 125 ug/ml were added and allowed to settle for 20 minutes. A rare-earth magnet was placed approximately 8 mm above the sensor surface for 60 seconds before measurement. The magnet position was determined empirically. The sample was then placed in the measurement system. FIG. 10 illustrates relative output signal magnitude vs. array element index illustrating generally how a digital magnetic bead detection can perform quantitation according to specific embodiments of the invention for an example anti-Hu IgG target protein (upper) and control (lower). In this example, the average signal-to-noise ratio is approximately 13 db. FIG. 10 shows the response from the first 96 sensor elements for the goat anti-human IgG shown in grey and the goat anti-mouse IgG shown in black.

This data was collected prior to the use of electrical modulation. Recent experiments indicate that use of electrical modulation improves SNR by approximately 10-20 dB. This improvement in SNR can be used to reduce the scan time for the array. Scanning all 256 elements takes approximately 2 minutes. The scanning time for the array becomes important if the device is scanned repeatedly while the magnetic washing force is ramped. This washing method may provide additional information about assay binding characteristics.

Thus, according to specific embodiments, the present invention provides an immunoassay platform with clinically relevant sensitivity, fabricated in a CMOS process. The noise and sensitivity limitations that traditionally limit the applicability of CMOS Hall sensors have been mitigated through architectural and signal processing techniques. With these improvements, the CMOS substrate is expected to provide a cost-effective and easily manufactured platform for diagnostics. Furthermore, the proposed assay platform is potentially compact and automated, making it applicable to in-field applications.

16. Example Applications

In specific embodiments, the invention can be embodied in an inexpensive, simple and robust assays using sensor technology as described herein to rapidly detect HIV virus and HIV-specific antibodies, for use in point-of-care diagnostic clinical settings. By directly integrating millimeter-sized computer chips with biological assays, the need for a laboratory, capital equipment and trained personnel can be eliminated. Due to the small size of the individual detectors, many tests can be simultaneously done on a drop of blood. In particular, in wireless embodiments, a variety of differently coated wireless sensors and appropriately coated paramagnetic beads can be added to a sample and provide simultaneous results from a very small blood or serum sample. In specific embodiments, existing ELISA technology can be transferred to an ImmunoSensor™ platform. In further embodiments, DNA probes that target invariant sequences of HIV genomic RNA can be used to quantitate viral RNA without amplification. In further embodiments, the invention can provide analysis of different parameters of infection including HIV-specific antibody, virus, and viral RNA by employing various chips in a single blood or serum specimen will be optimized.

In specific embodiments, sensors according to the invention are created on a sub-micron scale providing an “intelligent” substrate, capable of data-acquisition, data-processing, and communication in a physical space of 1 mm², and a cost of ˜25 cents. The chip surface is modified with a gold overlay to allow interaction with biological molecules which determine the disease specificity. Thus, while all the chips are manufactured in an identical fashion, they are subsequently treated with distinct biological molecules (antibody, antigen, DNA) that make them uniquely capable of detecting the presence of a particular pathogen or antibodies against that pathogen.

In specific systems, detection information is transmitted to a hand-held device, such as a PDA, as easily-interpretable numeric results. The use of a battery-operated PDA provides a simple and rapid read-out which will work in the absence of electricity for field use. The stability of the IC chips (no refrigeration) and biological reagents (minimal refrigeration) is a distinct advantage.

For HIV Antibody Detection for example sensor chips are manufactured in large batch format and then diced into 1 mm², chips, each with thousands of sensors. Chips can be coated with antigen or antibody specific for HIV gp120 and exposed to the test sample. HIV virus or gp120 protein adheres to the antibody on the sensor and specific protein-coated magnetic beads will bind and sandwich the target virus/protein. Magnetic beads that do not interact with the target protein are removed using a controlled magnetic force or other washing mechanism, enabling automated removal of non-specific binding. The sensor then measures the amount of bound magnetic beads, indicating presence of the target protein, and relays the information to the hand-held reader. This methodology has been demonstrated using a reference human IgG detection assay, and a clinical assay for Dengue infection.

In other example applications, HIV-specific DNA oligos are attached to the gold chip surface using thiol group-based linkers, blood samples in lysis buffer can be added to the chips, and complementary HIV viral RNA will bind to the oligos. Magnetic beads targeted to the bound viral RNA complex are also added, washed, and the results relayed to the PDA. Due to the inherent amplification of the magnetic bead signal, no additional amplification of the target or signal is necessary. A parallel assay will be developed to measure both HIV virus and antibody simultaneously. This methodology can be used to detect opportunistic infections as well.

In further embodiments, the invention provides a versatile platform technology that can be adapted to detect virtually any biological component to which there is a specific binding agent. Its small size allows for several differently coated chips to be place within a small fluid volume (i.e. a drop of blood) for simultaneous analysis.

As a further example, the present invention enables an effective portable system for collecting data regarding dengue, the most medically important mosquito-borne viral illness worldwide, with over 100 million cases annually. High through-put diagnostics are critical for management of the often explosive urban epidemics, and current cost and technical limitations hamper diagnostic efforts. Results to date have shown that anti-dengue virus (DEN) and anti-human IgG can be detected using a sensor package in a “flip-chip” format and can further detect anti-DEN IgG, anti-DEN IgM, and DEN antigen. A single, simultaneous assay that tests for DEN and Leptospira antibodies and antigens can be used for differential diagnosis and other contexts where exposure to multiple pathogens needs to be screened simultaneously.

Furthermore, wireless sensor chips are powered and interrogated remotely, using a wireless electromagnetic connection. Each chip can be tagged with an electronic ID, much like a telephone number, to allow distinction from other chips. The sensor chips will be added to the sample, and measured from outside the well. This approach eliminates the requirement of drying the coating protein, as the coated chips can remain in liquid continuously. Furthermore, this technology can significantly reduce the cost per assay as packaging and assembly of the device that normally increases cost are not required. Combination with a Palm Pilot or other handheld device provides a portable, simple, and reliable assay system will allow decentralization of testing in many developing countries.

In further embodiments, the invention enables improved HIV viral load detection. Many HIV viral load assays have traditionally been PCR-based, involving amplification to detect RNA. In contrast, inherent properties of a Hall sensor according to specific embodiments of the invention allow it to detect small quantities of RNA without amplification. The invention accomplishes this by increasing the sensitivity, which is governed by the signal-to-noise ratio. The sensitivity of the sensor allows a single bound bead—representing a single bound RNA—to be detected, whereas biological reporters require large numbers of elements to bind the target complex. Immunological assays rely on ligand-receptor interactions on the order of 250 pN/bond. A magnetic bead conjugated with oligonucleotides complementary to the target or other bound probes capitalizes on the superior strength of oligonucleotide base-pairing interactions, which is on the order of 10,000 pN for 20 bp. Taken together, the high binding affinity of an oligo-conjugated magnetic bead coupled with the sensitivity of the sensor allows direct, unamplified detection of target RNA.

In further specific embodiments, the use of a gold substrate for immobilizing “capture probes” presents unique opportunities for improving target RNA hybridization efficiency and kinetics. Previous work has demonstrated the critical importance of surface probe density on hybridization efficiency and hybridization kinetics of microarray-based applications. The hybridization efficiency imposes a boundary on the absolute sensitivity of a given RNA detection assay. Ionic strength and surface charge can be used to modify surface probe density and can easily be manipulated in a MEMS-based device. Furthermore, special gold-sulfur interactions may be specifically exploited to vary the surface probe density. Thiolated probes in a thiol-based solvent can be used to generate a self-assembling monolayer of capture probes, whose density can be easily manipulated by varying both the probe concentration in the mixture and the time that the gold substrate is exposed to the probe/thiol mixture. This approach not only allows for variation in surface probe density, but prevents non-specific nucleic acid adhesion to the substrate due to the blocking of available sites with the thiol solvent as capture probe immobilization occurs. FIG. 20 (Table 1) illustrates an example of diseases, conditions, or statuses for which at least one gene is differentially expressed that can evaluated according to specific embodiments of the present invention.

An array device, according to specific embodiments of the present invention, could include a number of ligands and the array could be used to detect which site a drug most bound to.

C1q as a Immunoassay Detection Reagent

According to specific embodiments of the invention, the invention can be used with specific binding agents, such as C1q. C1q is a primary component of complement comprised of 6 identical subunits with collagen-like tails that bind to the Fc regions of antibodies when the antibodies are bound to cognate antigen. The C1q molecule must bind to either 2 molecules of IgG or 1 molecule of IgM to initiate the activities of complement. An immunoassay according to specific embodiments of the invention benefits from the use of C1q because it requires bound antibody to get Fc binding. Therefore C1q will preferentially bind to antibodies that are bound to their antigen. Therefore, C1q can be used as a secondary detection reagent in immunoassays to provide specificity for detecting bound antibodies. This can reduce or eliminate the need for liquid washing to remove unbound antibodies present in a sample. Thus, according to specific embodiments of the invention, C1q conjugated to a magnetic bead can be used as a secondary regent that can attach to bound antibodies and provide the ability to determine the amount of antibodies bound to a surface antigen. The magnetic beads will be detected using the sensor described above. This will eliminate the need to provide liquid washing to remove serum samples containing unbound antibodies. In further embodiments, Biotinylated C1q can be used as a detection regent that can attach to bound antibodies and provide the ability to determine the amount of antibodies bound to a surface antigen. Streptavidin-coated magnetic beads can bind to the biotin on the C1q for detection and quantitation of bound antibodies.

17. Other Uses and Embodiments

Diagnostic Uses

As described above, following identification and validation of a detector for a particular substance, including biological molecules such as genes, proteins, sugars, carbohydrates, fats or any oligonucleotide or polypeptide of interest according to the invention, in specific embodiments such detectors are used in clinical or research settings, such as to predictively categorize subjects into disease-relevant classes. Detectors according to the methods the invention can be utilized for a variety of purposes by researchers, physicians, healthcare workers, hospitals, laboratories, patients, companies and other institutions. For example, the detectors can be applied to: diagnose disease; assess severity of disease; predict future occurrence of disease; predict future complications of disease; determine disease prognosis; evaluate the patient's risk; assess response to current drug therapy; assess response to current non-pharmacologic therapy; determine the most appropriate medication or treatment for the patient; and determine most appropriate additional diagnostic testing for the patient, among other clinically and epidemiologically relevant applications. Essentially any disease, condition, or status for which at least one gene is differentially expressed can be evaluated, e.g., diagnosed, monitored, etc. using the diagnostic gene sets and methods of the invention, see, e.g. Table 1.

In addition to assessing health status at an individual level, the methods and diagnostic sensors of the present invention are suitable for evaluating subjects at a “population level,” e.g., for epidemiological studies, or for population screening for a condition or disease. Expression profiles can be assessed in subject samples using the same or different techniques as those used to identify and validate the diagnostic sensors.

Web Site Embodiment

The methods of this invention can be implemented in a localized or distributed data environment. For example, in one embodiment featuring a localized computing environment, a sensor according to specific embodiments of the present invention is configured in proximity to a detector, which is, in turn, linked to a computational device equipped with user input and output features. In a distributed environment, the methods can be implemented on a single computer, a computer with multiple processes or, alternatively, on multiple computers. Sensors according to specific embodiments of the present invention can be placed onto wireless integrated circuit devices (e.g., “smart dust”) and such wireless devices can return data to a configured information processing system for receiving such devices. In the present invention, wireless “Smart Dust” implementations are practical because a wireless Hall Effect Magnetic Bead Sensor can be inductively powered and/or wirelessly read while in a reader wherein an inductive powering element and/or wireless reading element are very close (e.g., within 2-10 millimeters) of the magnetic bead sensors.

Kits

A detector according to specific embodiments of the present invention is optionally provided to a user as a kit. Typically, a kit of the invention contains one or more sensors constructed according to the methods described herein. Most often, the kit contains a diagnostic sensor packaged in a suitable container. The kit typically further comprises, one or more additional reagents, e.g., substrates, labels, primers, for labeling expression products, tubes and/or other accessories, reagents for collecting blood samples, buffers, e.g., erythrocyte lysis buffer, leukocyte lysis buffer, hybridization chambers, cover slips, etc., as well as a software package, e.g., including the statistical methods of the invention, e.g., as described above, and a password and/or account number for accessing the compiled database. The kit optionally further comprises an instruction set or user manual detailing preferred methods of using the kit components for sensing a substance of interest.

When used according to the instructions, the kit enables the user to identify disease specific substances (such as genes and/or proteins and/or sugars and/or viruses and/or antibodies and/or other anti-gens) using patient tissues, including, but not limited to blood. The kit can also allow the user to access a central database server for example using a wireless or satellite telephone that receives and/or provides expression information to the user. Such information can facilitates the discovery of additional diagnostic gene sets by the user or facilitate wide ranging public health management programs in areas with limited technical and/or communication infrastructure. Additionally, or alternatively, the kit allows the user, e.g., a health care practitioner, clinical laboratory, or researcher, to determine the probability that an individual belongs to a clinically relevant class of subjects (diagnostic or otherwise).

Embodiment in a Programmed Information Appliance

The invention may be embodied in whole or in part within the circuitry of an application specific integrated circuit (ASIC) or a programmable logic device (PLD). In such a case, the invention may be embodied in a computer understandable descriptor language, which may be used to create an ASIC, or PLD that operates as herein described.

Integrated Systems

Integrated systems for the collection and analysis of expression profiles, molecular signatures, as well as for the compilation, storage and access of the databases of the invention, typically include a digital information appliance (e.g., a PDA or portable computer) with software including an instruction set for sequence searching and/or analysis, and, optionally, one or more of high-throughput sample control software, image analysis software, data interpretation software, a robotic control armature for transferring solutions from a source to a destination (such as a detection device) operably linked to the digital computer, an input device (e.g., a computer keyboard) for entering subject data to the digital computer, or to control analysis operations or high throughput sample transfer by the robotic control armature.

Readily available computational hardware resources using standard operating systems can be employed and modified according to the teachings provided herein, e.g., a PC or PDA (Intel x86 or Pentium chip-compatible DOS,™ OS2,™ WINDOWS,™ WINDOWS NT,™ WINDOWS95,™ WINDOWS98,™ LINUX, or even Macintosh, Sun or PCs will suffice) for use in the integrated systems of the invention. Current art in software technology is adequate to allow implementation of the methods taught herein on a computer system. Thus, in specific embodiments, the present invention can comprise a set of logic instructions (either software, or hardware encoded instructions) for performing one or more of the methods as taught herein. For example, software for providing the described data and/or statistical analysis can be constructed by one of skill using a standard programming language such as Visual Basic, Fortran, Basic, Java, or the like. Such software can also be constructed utilizing a variety of statistical programming languages, toolkits, or libraries.

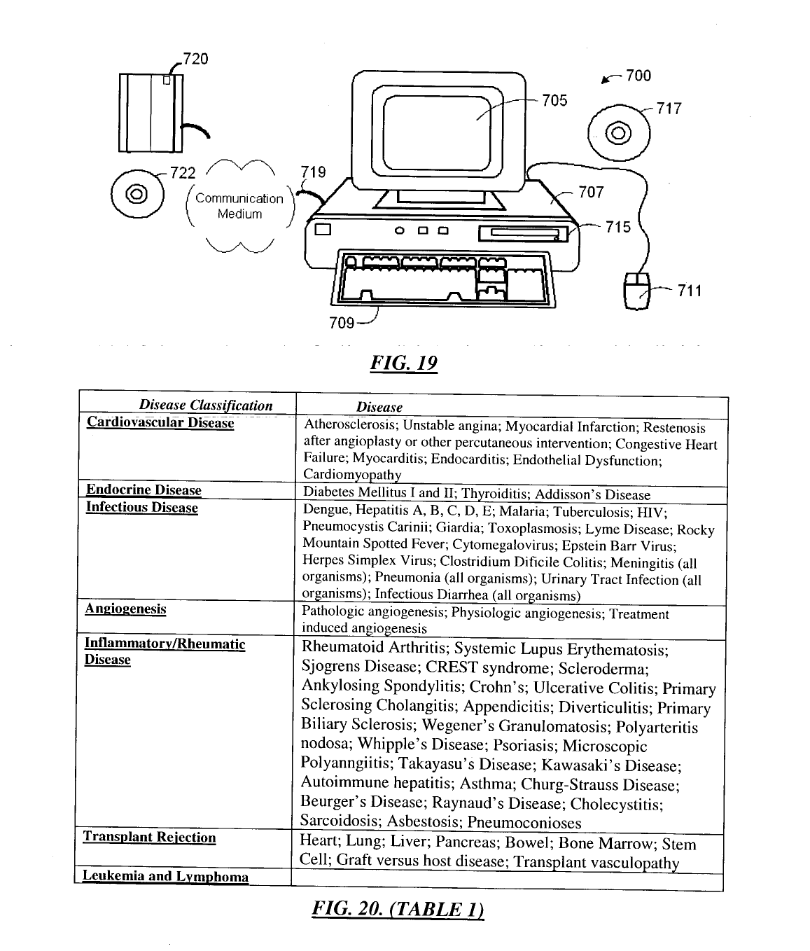

FIG. 19 is a block diagram showing a representative example logic device in which various aspects of the present invention may be embodied. FIG. 19 shows an information appliance (or digital device) 700 that may be understood as a logical apparatus that can read instructions from media 717 and/or network port 719, which can optionally be connected to server 720 having fixed media 722. Apparatus 700 can thereafter use those instructions to direct server or client logic, as understood in the art, to embody aspects of the invention. One type of logical apparatus that may embody the invention is a computer system as illustrated in 700, containing CPU 707, optional input devices 709 and 711, disk drives 715 and optional monitor 705. Fixed media 717, or fixed media 722 over port 719, may be used to program such a system and may represent a disk-type optical or magnetic media, magnetic tape, solid state dynamic or static memory, etc. In specific embodiments, the invention may be embodied in whole or in part as software recorded on this fixed media. Communication port 719 may also be used to initially receive instructions that are used to program such a system and may represent any type of communication connection. Another type of device preferable in specific embodiments is a hand-held information appliance, such as a Personal Digital Assistant (PDA) that can-be programmed to perform one or more of the data collection and/or data analysis methods as herein described.

Various programming methods and algorithms, including genetic algorithms and neural networks, can be used to perform aspects of the data collection, correlation, and storage functions, as well as other desirable functions, as described herein. In addition, digital or analog systems such as digital or analog computer systems can control a variety of other functions such as the display and/or control of input and output files. Software for performing the electrical analysis methods of the invention are also included in the computer systems of the invention.

Other Embodiments

Although the present invention has been described in terms of various specific embodiments, it is not intended that the invention be limited to these embodiments. Modification within the spirit of the invention will be apparent to those skilled in the art. In addition, various different actions can be used to effect a request for sequence data. For example, a voice command may be spoken by the purchaser, a key may be depressed by the purchaser, a button on a client-side scientific device may be depressed by the user, or selection using any pointing device may be effected by the user.

It is understood that the examples and embodiments described herein are for illustrative purposes and that various modifications or changes in light thereof will be suggested by the teachings herein to persons skilled in the art and are to be included within the spirit and purview of this application and scope of the claims.

All publications, patents, and patent applications cited herein or filed with this application, including any references filed as part of an Information Disclosure Statement, are incorporated by reference in their entirety.

What is claimed:

1. A method of detecting one or more substances of interest comprising:
exposing said one or more substances of interest to an integrated circuit Hall effect detecting device, said device coated with one or more molecules able to attach to said one or more substances of interest;
exposing said device to a plurality of magnetic beads, said magnetic beads configured to attach to said one or more substances of interest attached to said integrated circuit detecting device; applying a field parallel to said device, said field inducing perpendicular fields in said magnetic beads;
observing, in said integrated circuit, said magnetic beads using said induced perpendicular field; and
using said detecting to signal the presence of said one or more substances of interest.

2. The method according to claim 1 further comprising:
prior to exposing said device to a plurality of magnetic beads;
exposing said device to a plurality of different molecules, each molecule targeting one of said substances of interest at one end and each molecule having a common attachment for said magnetic beads.

3. The method according to claim 1 further wherein:
said observing comprises perceiving a deflected current through said integrated circuit device.

4. The method according to claim 1 further wherein:
said integrated circuit device comprises an active device.

5. The method according to claim 1 further wherein:
said integrated circuit device comprises a Hall effect transistor.

6. The method according to claim 1 further wherein:
said integrated circuit device comprises micron scale Hall effect sensors.

7. The method according to claim 1 further wherein:
said integrated circuit device comprises dual transistors with shared split drains, wherein source current flows in opposite directions in each transistor.

8. The method according to claim 1 further wherein:
said integrated circuit device comprises dual Hall sensors, wherein a Hall signal flows in opposite directions in each sensor in response to a global magnetic field.

9. An FET integrated circuit device comprising:
two channel regions, each with its own gate,
two separate source regions,
a shared drain region, configured such that current between said shared drain region will flow in opposite directions to said two source regions.

10. The device according to claim 9 further wherein:
said two channel regions are designed to be of roughly equal size.

11. The device according to claim 9 further wherein:
said device is designed so that in the absence of any deflecting field, the current in each channel when the source voltages and gate voltages are equal will be of the same magnitude.

12. The device according to claim 9 further wherein:
said shared drain is a split drain, with one portion situated at one said of said two channels and the other portion situated at the other side of said channel.

13. An FET integrated circuit device comprising:
means for two channel regions;
two gate means for said two channel regions,
means for two separate source regions,
means for a shared drain region, configured such that current between said shared drain region will flow in opposite directions to said two source regions.

14. A detector for one or more substances of interest comprising:
means for exposing said one or more substances of interest to an integrated circuit detecting device, said device coated with one or more molecules able to attach to said one or more substances of interest;
means for exposing said device to a plurality of magnetic beads, said magnetic beads configured to attach to said one or more substances of interest attached to said integrated circuit detecting device;
means for applying a field parallel to said device, said field inducing perpendicular fields in said magnetic beads;
means for observing, in said integrated circuit, said magnetic beads using said induced perpendicular field; and
means for using said detecting to signal the presence of said one or more substances of interest.

15. The device according to claim 14 further comprising:
means for exposing said device to a plurality of different molecules, each molecule targeting one of said substances of interest at one end and each molecule having a common attachment for said magnetic beads.

16. The method according to claim 1 further comprising:
separately addressing paired magnetic detectors using at least one gate voltage to selectively activate a paired detector.

17. The method according to claim 16 further comprising:
determining a quantitation for a target of interest by summing positive results from addressed detectors.

18. The method according to claim 1 further comprising:
scaling each one of paired magnetic detectors to be on the order of the diameter of a magnetic bead used for detection.

19. The method according to claim 1 further comprising:
using row and column addressing to rotate the drive and detection contacts of a four-contact Hall Device in order to provide an improved detection result.

20. The method according to claim 1 further wherein:
said magnetic bead detector that can be manufactured using standard integrated circuit (IC) fabrication processes.

21. A disposable assay sample system comprising:
a holder with a well for holding a sample of interest;
a plurality of magnetic beads, each able to specific bind to a substance that may be present in said sample of interest;
at least one array of magnetic bead sensors, each sensor able to detect the presence of one bound bead, said array coated with a specific binding molecule; and
circuitry connected with said array able to addressable transfer data from said array indicating detection results of sensors in said array;
wherein said holder is configured to be able to fit within a reader for reading said data and wherein said magnetic beads and a binding surface of said array are able to be arranged to make operative contact with said sample.

22. The system of claim 21 further comprising:
circuit means for connecting said array to said well in a fixed fashion; and
conductors on said holder for making electrical contact with said reader when said holder is placed therein.

23. The system of claim 21 further comprising:
wireless transmission circuitry integrated with said array; and
further wherein said array and said wireless circuits are not fixed to said holder and may be selectively introduced with a sample of interest.

24. A method of performing biologic and/or medical assays in areas in particular that have little or no technological infrastructure comprising:
transferring a sample to be tested to a disposable carrier;
introducing one or more magnetically active specific binding labels to said sample and configuring said sample and said labels to be adjacent to a biologically active integrated circuit magnetic detector;
reading data from said detector using a portable reader; and
transferring data from said portable reader to a standard portable information appliance and thereafter using said standard portable information appliance to record clinical results and communicate clinical data.

25. The method according to claim 24 further comprising:
using said standard portable information appliance to perform signal processing to determine results from said sensor.

26. The method according to claim 1 further comprising:
conjugating C1q to a magnetic bead as a secondary regent that can attach to bound antibodies and provide the ability to determine the amount of antibodies bound to a surface antigen.

27. The method according to claim 1 further comprising:
using biotinylated C1q as a detection regent that can attach to bound antibodies and provide the ability to determine the amount of antibodies bound to a surface antigen.

28. A method of detecting the presence of a magnetic bead of less than 20 microns in diameter comprising:
arranging a dual Hall Effect Sensor such that it will be proximate to an area where it is wished to detect the presence of a magnetic bead;
exposing said sensor to a magnetic field parallel to a plane of current flow of said sensor;
detecting the presence of a magnetic bead by measuring a difference in Hall signal flowing through each device in said dual Hall Effect sensor.
